(19) United States
(12) Patent Application Publication
(10) Pub. No.: US 2022/0081715 A1
(43) Pub. Date: Mar. 17, 2022

(54) mRNA-BASED BIOMARKERS FOR ANTIBODY-MEDIATED TRANSPLANT REJECTION

(71) Applicants: KATHOLIEKE UNIVERSITEIT LEUVEN, Leuven (BE); INSTITUT NATIONAL DE LA SANTE ET DE LA RECHERCHE MEDICALE, Paris (FR); COMMISSARIAT A L'ENERGIE ATOMIQUE ET AUX ENERGIES ALTERNATIVES, Paris (FR); MEDIZINISCHE HOCHSCHULE HANNOVER, Hannover (DE); UNIVERSITE DE PARIS, Paris (FR); CENTRE NATIONAL DE LA RECHERCHE SCIENTIFIQUE, Paris (FR); UNIVERSITE DE LIMOGES, Limoges (FR); LE CENTRE HOSPITALIER ET UNIVERSITAIRE DE LIMOGES, Limoges (FR)

(72) Inventors: Maarten NAESENS, Keerbergen (BE); Elisabet VAN LOON, Heverlee (BE); Pierre MARQUET, Limoges (FR); Wilfried GWINNER, Hannover (DE); Dany ANGLICHEAU, Paris (FR); Marie ESSIG, Limoges (FR); Stephane GAZUT, Gif-sur-Yvette (FR)

(21) Appl. No.: 17/420,347

(22) PCT Filed: Jan. 16, 2020
(86) PCT No.: PCT/EP2020/050959

§ 371 Date: Jul. 1, 2021

(30) Foreign Application Priority Data
Jan. 17, 2019 (EP) 19152365.3

(51) Int. Cl. C12Q 1/6876; C12Q 1/6851; C12Q 1/686

(52) U.S. Cl. CPC C12Q 1/6876; C12Q 1/6851; G01N 2800/245; C12Q 2600/158; C12Q 1/686

(57) ABSTRACT

The invention relates to methods for diagnosing or determining in a subject who underwent a solid organ transplantation the risk of developing graft rejection other than T cell mediated, comprising the steps of: measuring in a peripheral blood sample from said subject the RNA expression levels of a set of genes comprising at least CXCL10, FCGR1A, FCGR1B and TEMP1; determining from the measured expression levels of said set of genes, an expression profile; comparing the determined expression profile of said subject with a reference expression profile; and determining from said comparison, whether said subject is experiencing said rejection or has an increased risk of developing said rejection.

### FIELD OF THE INVENTION

The invention relate to methods and kits to predict the likelihood of a transplant rejection.

### BACKGROUND

Antibody-mediated rejection is recognized as a primary cause of graft failure after kidney transplantation. It is hallmarked histologically by inflammation and C4d deposition in peritubular capillaries, glomerulitis, intimal arteritis and expansion/duplication of the glomerular basement membrane [Haas et al. (2018) Am J Transplant 18, 293-307].

Currently, the diagnosis of antibody-mediated rejection after kidney transplantation is made based on histological assessment of invasive kidney biopsies according to the regularly updated Banff international consensus in patients with donor-specific antibodies or with signs of antibody activity [Haas et al. (2008) Am J Transplant 18, 293-307]. Antibody-mediated rejection can be diagnosed in clinically indicated biopsies at time of graft functional problems (rise in serum creatinine or proteinuria), but can also occur subclinically, without changes in these graft functional parameters. Subclinical antibody-mediated rejection also associates with increased risk of graft failure [Loupy et al. (2015) J Am Soc Nephrol 26, 1721-1731] but often remains undetected, unless protocol-specified kidney biopsies are performed. Such protocol-specified biopsies are routinely performed in some centres, but not all, at varying time after transplantation.

Given the association between antibody-mediated rejection and kidney graft failure, and the impossibility to repeatedly perform invasive protocol-specified biopsies, non-invasive diagnostic markers are needed with better sensitivity and specificity than eGFR and proteinuria [Loupy et al. (2015) J Am Soc Nephrol 26, 1721-1731]. Other groups have suggested non-invasive markers for antibody-mediated rejection, primarily assessed in urine samples [Blydt-Hansen et al. (2017) Transplantation 101, 2553-2561; Rabant et al. (2015) J Am Soc Nephrol 26, 2840-2851; Matignon et al. (2014) J Am Soc Nephrol 25, 1586-1597; Veale et al. (2006) Hum Immunol 67, 777-786; Ashton-Chess et al. (2008) J Am Soc Nephrol 19, 1116-1127; Slavcev et al. (2016) Arch Immunol Ther Exp (Warsz) 64, 47-53.]

As these markers have not been validated sufficiently, very few of them, if any, will be implemented in clinical practice [Naesens & Anglicheau (2017) J Am Soc Nephrol 29, 24-34].

Kidney allograft rejection is associated with molecular changes in renal allograft biopsies, which reflect transcription changes in resident cells (e.g. interferon-gamma inducible changes in the donor endothelium) or changes in cell populations, like infiltration and activation of effector T cells and macrophages in T-cell mediated rejection or margination and activation of natural killer cells in antibody-mediated rejection [Halloran et al. (2017) Am J Transplant 18, 785-795]. As these graft infiltrating cells are activated primarily in lymphoid organs before travelling and infiltrating the allograft [Nankivell & Alexander (2010) N Engl J Med. 363, 1451-1462], molecular changes that occur in renal allograft biopsies with antibody-mediated rejection could also be reflected by changes in circulating immune cells. WO 2015179777 discloses genome-wide gene analysis of expression profiles of over 50,000 known or putative gene sequences in peripheral blood, to identify a subclinical acute rejection (subAR).

EP 3146077 discloses markers in a kidney biopsy that determine patients who have Acute Rejection (AR), Acute Dysfunction No Rejection (ADNR), Chronic Allograft Nephropathy (CAN), or Transplant Excellent/Normal (TX) condition.

There is nevertheless a need for more reliable and non-invasive methods to monitor blood samples of patients who underwent kidney transplantation.

Given the lack of non-invasive markers for antibody-mediated rejection, there is a need to develop and validate an mRNA-based gene set in peripheral blood that is able to non-invasively rule out or detect ongoing antibody-mediated rejection after kidney transplantation.

### SUMMARY OF THE INVENTION

A novel 8-gene expression assay in peripheral blood was developed and validated that can be used for non-invasive diagnosis of antibody-mediated rejection of kidney allografts.

In the discovery and derivation phases, a multigene assay of 8 genes was developed and locked (CXCL10, GBP1, IL15, FCGR1A, FCGR1B, GBP4, KLRC1, TIMP1) in peripheral blood that discriminated cases with (N=49) from cases without (N=134) antibody-mediated rejection (diagnostic accuracy in the derivation cohort, 78.1% (95% confidence interval [CI], 70.7 to 85.6). In the independent validation cohort, this 8-gene marker discriminated cases with (N=41) from cases without antibody-mediated rejection (N=346) with similar accuracy (79.9%; 95% CI, 72.6 to 87.2). The 8-gene assay retained accuracy for antibody-mediated rejection in patients with stable graft function (83.4%; 95% CI, 75.4 to 91.3) and at time of graft dysfunction (75.3% 95% CI, 64.9 to 85.8), within the first year (90.9%; 95% CI, 85.3 to 96.4) and also later after transplantation (73.5%; 95% CI, 63.6 to 83.4). Integration of the 8-gene assay with data on donor-specific antibodies, proteinuria and estimated glomerular filtration rate further increased the diagnostic accuracy (87.8%; 95% CI, 82.6 to 93.0), and provided net benefit for clinical decision-making.

The invention is further summarized in the following statements:

1. A method for diagnosing or determining in a subject who underwent a solid organ transplantation the risk of developing graft rejection other than T cell mediated, the method comprising the steps of:
measuring in a peripheral blood sample, or in a biopsy of the transplant, from said subject the RNA expression levels of a set of genes comprising at least CXCL10, FCGR1A, FCGR1B and TIMP1,
determining from the measured expression levels of said set of genes, an expression profile,
comparing the determined expression profile of said subject with a reference expression profile,
determining from said comparison, whether said subject is experiencing said rejection or has an increased risk of developing said rejection.
   2. A method for distinguishing, in a subject who underwent a solid organ transplantation, between antibody mediated rejection and T cell mediated rejection, comprising the steps of:
measuring in a peripheral blood sample, or in a biopsy of the transplant, from said subject the RNA expression level of a set of genes comprising at least CXCL10, FCGR1A FCGR1B and TIMP1,
comparing the measured RNA expression levels of each genes in the set with the RNA expression level of at least one reference housekeeping gene,
   wherein a high expression level of CXCL10 and FCGR1A compared to the expression level of the least one reference gene, and wherein a low expression level of FCGR1B and TIMP1, compared to the expression level of the least one reference gene is indicative of antibody mediated rejection.
   3. The method according to statement 1 or 2, wherein the set of genes further comprises one or more of GBP4, KLRC1, GBP1 and IL 15.
   4. The method according to any of statements 1 to 3, wherein measured expression levels are normalised against housekeeping genes.
   5. The method according to any one of statements 1 to 4, wherein the graft rejection other than T cell mediated rejection, is selected from the group consisting of antibody mediated rejection, changes suspicious of antibody-mediated rejection, and mixed T cell and antibody mediated rejection.
   6. The method according to statement any one statements 1 to 5, wherein the graft rejection other than T cell mediated rejection is antibody mediated rejection.
   7. The method according to any one of statements 1 to 6, wherein the solid organ transplant is a kidney transplant.
   8. The method according to any one of statements 1 to 7, wherein, for the calculation of the expression profile a weight factor is attributed to the quantitative expression level of each gene, where the weight factor of CXCL10<GBP4<KRLC1<FCGR1A and wherein the weight factor GBP1<FCGR1B<IL15<TIMP1.
   9. The method according to statement 8, wherein the weight factor of GBP1<CXCL10<GBP4<KRLC1<FCGR1B<IL15<TIMP1<FCGR1A.
   10. The method according to any one of statements 1 to 9, wherein a linear regression algorithm is used to compare the expression profile of said subject with a reference expression profile.
   11. The method according to statement 10, wherein the linear regression algorithm is represented by the formula

log(odds ABMR)=“constant value”+a*ΔCq_CXCL10+b*ΔCq_GBP1+c*ΔCq_IL15+d*ΔCq_FCGR1A+e*ΔCq_FCGR1B+f*ΔCq_GBP4+g*ΔCq_KLRC1+h*ΔCq_TIMP1,

wherein a value of log (odds ABMR) typically above −0.83 is indicative of rejection other than T cell mediated rejection,
wherein “constant value” is −2.52±0.25, a is −0.25±0.025, b is 0.086±0.0086, c is 0.76±0.076, d is −1.21±0.12, e is +0.45±0.045, f is −0.31±0.031, g−0.40±0.04 and is h is 1.03±0.1,
and wherein ΔCq of a gene corresponds to the mean delta-Cq of each gene, which is the difference between the measured Cq value of each gene and the mean Cq value of three housekeeping genes.
12. The method according to statement 11, wherein “constant value” is −2.52±0.126, a is −0.25±0.013, b is 0.086±0.004, c is 0.76±0.038, d is −1.21±0.061, e is +0.45±0.023, f is −0.31±0.016, g−0.40±0.020 and is h is 1.03±0.052.
13. The method according to any one of statements 1 to 12, wherein the RNA expression levels are determined by a quantitative PCR amplification method.
14. The method according to any one of statements 1 to 13, which is performed within 12 months after transplantation.
15. The method according to any one of statements 1 to 14, which is performed on subjects with stable graft function or non stable graft function.
16. The method according to any one of statements 4 to 15, wherein the housekeeping genes comprise one or more of ACTB, GAPDH and SDHA.
17. The method according to any one of statements 1 to 16, further comprising the step of one or more of identifying presence or absence of donor-specific antibodies, proteinuria and estimated glomerular filtration rate.
18. Use of nucleic acid probes for determining the RNA expression set of genes in detecting the increased risk, development or presence of developing antibody mediated rejection in a subject who underwent kidney transplantation or another solid organ transplantation, wherein the set of genes comprising at least CXCL10, FCGR1A FCGR1B and TIMP1.
19. The use according to statement 18, wherein the set genes further comprises, one or more of GBP4, KLRC1, GBP1 and IL 15.
20. A kit for in vitro diagnosis of solid organ graft rejection other than T cell mediated rejection comprising:
(i) a probe or set of probes for determining the expression level of at least CXCL10, FCGR1A FCGR1B and TIMP1, and optionally the expression level of GBP4, KLRC1, GBP1 and IL 15
(ii) a probe or set of probes for determining the expression level of housekeeping genes,
   wherein the kit comprises no more than 50 probes or set of probes for determining the expression level of a gene that does not belong to the genes of (i) and that is not a housekeeping gene. In other embodiments such kit comprises, no more than 40, 25 or 10 probes or set of probes for determining the expression level of a gene that does not belong to the genes of (i) and that is not a housekeeping gene.
   The kit according to the above statement wherein the solid organ graft is a kidney.
   21. The kit according to statement 20, further comprising instructions for use.
   22. The kit according to statement 21, wherein the instructions comprise a formula for calculating an expression profile, and calculating the odds of rejection other than T cell mediated rejection.
   23. A kit for in vitro diagnosis of kidney rejection other than T cell mediated rejection comprising:
   (i) a nucleic acid probe or set of nucleic acid probes, for determining the expression level of at least CXCL10, FCGR1A FCGR1B and TIMP1, and optionally a nucleic acid probe or set of nucleic acid probes for determining the expression level of GBP4, KLRC1, GBP1 and IL 15, and
   (ii) optionally a nucleic acid probe or set of nucleic acid probes for determining the expression level of housekeeping genes,
   wherein the kit comprises no more than 50 nucleic acid probess for determining the expression level of a gene that does not belong to the genes of (i) and that is not a housekeeping gene,
   or
   wherein the kit comprises no more than 50 sets of nucleic acid probes for determining the expression of gene that does not belong to the genes of (i) and that is not a housekeeping gene.

The kit can further comprise probes or set of probes for determining the expression of one or more of the following genes:

CFLAR (CASP8 and FADD like apoptosis regulator), DUSP1 (dual specificity phosphatase 1), IFNGR1 (interferon gamma receptor 1), ITGAX (integrin subunit alpha X), MAPK9 (mitogen-activated protein kinase 9), NAMPT (nicotinamide phosphoribosyltransferase), NKTR (natural killer cell triggering receptor), PSEN1 (presenilin 1), RNF130 (ring finger protein 130), RYBP (RING1 and YY1 binding protein, CEACAM4 (carcinoembryonic antigen related cell adhesion molecule 4), EPOR (erythropoietin receptor), GZMK (granzyme K), RARA (retinoic acid receptor alpha), RHEB (Ras homolog, mTORC1 binding), RXRA (retinoid X receptor alpha), SLC25A37 (solute carrier family 25 member 37). The expression of these genes can be used to detect renal transplant patients at high risk for acute rejection of a solid organ transplant, in particular of a kidney transplant. The method for detecting acute rejection based on these markers is described in Roedder et al. (2014) PLoS Med. 11, e1001759.

### DETAILED DESCRIPTION

### Description of the Figures

FIG. 1. Enrolment and sample distribution. Peripheral blood samples were obtained at the time of a renal allograft biopsy in four European transplant centres. In the discovery and derivation cohort, samples were selected based on availability and histological criteria of concomitant renal allograft biopsies (excluding cases with diagnosis of glomerulonephritis or polyomavirus nephropathy, and cases with unclear diagnosis), while graft function was not taken into account. In the validation cohort, all samples with concomitant adequate renal allograft biopsy histology, prospectively collected between Jun. 24, 2014 and Jul. 2, 2015, were serially included without selection on histology, demographics or time. The gene expression profile was not complete in seven of these samples, leading to a total of 387 cases in the validation phase.

FIG. 2. Diagnostic accuracy of the 8-gene assay according for non-invasive diagnosis of antibody-mediated rejection. The left panels show the 8-gene assay score in cases with versus without antibody-mediated rejection, in the derivation cohort (Panel A) and the validation cohort (Panel B). The middle panels show the distribution of cases with antibody-mediated rejection across all scores of the 8-gene assay. The right panels show the ROC curves for samples with versus without antibody-mediated rejection, with presentation of the area under the curve (AUC) and the 95% confidence interval.

FIG. 3. Diagnostic accuracy of the 8-gene assay in specific subgroups and combination with routine clinical parameters. Sensitivity analysis of the 8-gene marker in subgroups is shown in panel A and B. Panel C shows the diagnostic performance of clinical parameters (proteinuria (g/g creatinine), donor-specific antibodies (DSA) and estimated glomerular filtration rate (MDRD, ml/min/1.73 m²) represented by the Receiver Operating Characteristic (ROC) curves and area under the curve (AUC). To note: diagnostic performance of clinical parameters is not optimism corrected (model built on Phase 3).

FIG. 4. Pathway enrichment analysis for the gene lists assessed in the discovery cohort. Two gene lists were determined based on their ABMR and TCMR scores in blood and biopsy samples. This graph illustrates the canonical pathway enrichment analysis assessed with Ingenuity Pathway Analysis for each gene list. The first gene list consisted of the 44 probe sets (38 individual genes with ABMR score >0.25 in both biopsies and peripheral blood samples (Panel A). The second gene list consisted of 104 probe sets (79 genes) with ABMR score >0.25 in biopsies and >0.20 in peripheral blood (Panel B). P values are −log 10 transformed. Significance (p<0.05) is marked by the dotted line.

FIG. 5. Univariate associations of 44 gene transcripts with rejection phenotypes in the derivation cohort. In the derivation cohort (N=183), univariate analysis was performed for the 44 selected transcripts of the restricted list, for different types of rejection. Overview of pairwise comparison p-values (−log 10 transformed) is shown for each gene (N=44) in the comparison between presence (N=49) and absence of antibody-mediated rejection (N=134), between antibody-mediated rejection and T-cell mediated rejection, and between presence and absence of T-cell mediated rejection. Significance is represented by dotted lines at p<0.05 and p<0.001.

FIG. 6. Distribution of the 8-gene score according to rejection types in the validation cohort (N=387). Panel A shows distribution of the 8-gene score in antibody-mediated rejection versus T-cell mediated rejection versus absence of rejection. Cases with mixed rejection (N=3) were not included in this analysis. Panel B shows distribution for samples with versus without T-cell mediated rejection. Significant differences are represented (p<0.05; assessed with unpaired T test).

FIG. 7. Distribution of the 8-gene assay score per histological lesion grade in the validation cohort (N=387). The 8-gene assay score was significantly associated with lesions of antibody-mediated rejection. (panel A: glomerulitis, peritubular capillaritis, microvascular inflammation, transplant glomerulopathy; Panel B: interstitial inflammation, tubulitis, intimal arteritis, C4d capillary deposition; Panel C: interstitial fibrosis, tubular atrophy, intimal fibrosis, arteriolar hyalinosis). Significance was assessed with nonparametric one-way ANOVA. Significance was apparent for higher severity grades of lesions associated with antibody-mediated rejection (glomerulitis, peritubular capillaritis, microvascular inflammation score, transplant glomerulopathy and intimal arteritis). No significant association was present with lesions of T-cell mediated rejection (tubulitis, interstitial inflammation) or non-specific chronic damage (interstitial fibrosis and tubular atrophy). g=glomerulitis; ptc=peritubular capillaritis; mvi=microvascular inflammation; cg=transplant glomerulopathy; i interstitial inflammation; t=tubulitis; v=intimal arteritis; C4d=C4d deposition in peritubular capillaries; ci=interstitial fibrosis; ct=tubular atrophy; cv=intimal fibrosis; ah=arteriolar hyalinosis.

FIG. 8. Accuracy metrics according to the 8-gene score, as observed in the validation cohort (N=387). Test parameters of the 8-gene assay for diagnosis of antibody-mediated rejection were assessed in the validation cohort (N=387 samples). [Part A: specificity, sensitivity and Youden index. Part B: NPV, PPV and probability]. Evolution of these accuracy metrics according to the 8-gene score is shown here. The optimal cut-off (at highest Youden index; −0.83) and arbitrarily selected cut-offs for positive (1.04) and negative test (−1.32) are marked by dotted lines. Youden index=maximum summation of sensitivity and specificity minus 1. PPV=positive predictive value. NPV=negative predictive value.

FIG. 9. Decision curve analysis for the 8-gene assay in the validation set (N=387). Decision curve analysis evaluates the utility of the 8-gene assay to decide who should receive invasive diagnostic intervention (i.e. biopsy). Net benefit represents the proportion of “net” true positives in the dataset: the observed number of true positives is corrected for the observed proportion of false positives weighted by the odds of the risk threshold and the result is divided by the sample size (30). The X axis shows the range of threshold probabilities of antibody-mediated rejection (range 5-35%). We chose an upper limit of 35% because though doctors (taking into account patient preferences) might vary in their values for finding antibody-mediated rejection with avoiding unnecessary biopsies, it is unrealistic that any doctor or patient would need more than a 35% risk of antibody-mediated rejection before a biopsy is recommended. The Y axis shows the net benefit for decision-making. The net benefit line of the ‘Biopsy All’ strategy crosses the Y axis at the prevalence. The two default strategies without use of the assay are to biopsy all patients or biopsy none. The net benefit of biopsy none is always 0 because this strategy has no true or false positives. For risk thresholds below the prevalence, the biopsy all strategy has a higher net benefit than biopsy none. For thresholds above the prevalence, the opposite is true. The 8-gene assay has a higher net benefit than the default strategies in the defined threshold ranges. This implies that the 8-gene assay carries a net benefit for decision-making (whether or not to perform a biopsy) in the defined range of threshold probabilities.

FIG. 10. Flow diagram for the 8-gene assay according to the STARD guideline, in the validation cohort (N=387). We defined a positive test as a value above the optimal cut-off (−0.83) and a negative test when the value of the score was below this cut-off. The standard reference test for diagnosis of antibody-mediated rejection was histology. The 8-gene assay was not available in 7 of the 394 samples. The STARD diagram illustrates the clinical utility of our marker. A negative test is associated with a high negative predictive value (96.9%); only 8 cases with antibody-mediated rejection are classified as false negative. A positive test has a positive predictive value of 25.0%; i.e. a risk of one in four to have antibody-mediated rejection, which in clinical practice justifies performing an invasive biopsy.

FIG. 11: Diagnostic accuracy of the minimal signature expression profile containing 4 genes. Receiver Operating Characteristic (ROC) curve with 95% confidence bounds for the 4-gene expression profile in diagnosis of ABMR vs no ABMR in the derivation cohort (N=183) is shown.

### DEFINITIONS

### TCMR Refers to T-Cell Mediated Rejection.

The incidence of TCMR, which had a major impact on graft function and survival early after transplantation in the previous decades, has significantly decreased since application of more efficacious immunosuppressive regimens with tacrolimus, mycophenolate mofetil and induction therapy [Nankivell B J & Alexander (2010) N Engl J Med. 363, 1451-1462]. Nevertheless, the existing immunosuppressive armamentarium is insufficient in preventing patients from developing humoral alloreactivity, with the occurrence of circulating donor-specific HLA antibodies (DSAs) and ABMR [Nankivell & Alexander, cited above; Loupy et al. (2012) Nat Rev Nephrol. 8, 348-357; Djamali et al. (2014) Am J Transplant. 14, 255-271; Amore (2015) Curr Opin Organ Transplant. 20, 536-542]. In recent years, DSAs were demonstrated to be a crucial prognostic factor for graft outcome, and ABMR is now recognized as a prime reason for graft failure after kidney transplantation [Naesens et al. (2014) Transplantation. 98, 427-435; EI-Zoghby et al. (2009) Am J Transplant. 9, 527-535; Sellarés et al. (2012) Am J Transplant. 12, 388-399; Naesens et al. (2016) J Am Soc Nephrol. 27, 281-292].

### ABMR Refers to Antibody-Mediated Rejection.

ABMR is often mediated by antibodies directed against allogeneic major histocompatibility complex (MHC) molecules via the complement system. MHC molecules are interchangeably referred to as human leukocyte antigens (HLAs). HLAs are responsible for allorecognition, and without immunosuppression, allografts from a donor with different HLAs will be rejected. There are more than 1600 alleles for HLA class I and II molecules [Colvin & Smith (2005) Nat Rev Immunol 10, 807-817; Mandelbrodt & Mohamed Transplantation immunobiology. In: anovitch, ed. Handbook of Kidney Transplantation. Philadelphia, Pa.: Lippincott Williams & Wilkins (2010) 19-23.]. HLA class I molecules (e.g., HLA-A, HLA-B, HLA-C) are found on all nucleated cells, but HLA class II molecules (e.g., HLA-DP, HLA-DQ, HLA-DR) are expressed only on antigen-presenting cells (APCs). Among a recipient's anti-HLA antibodies, those specific to the donor's HLAs are DSAs. Less frequently, antibodies against other antigenic stimulants, such as ABO blood group antigens, minor histocompatibility antigens, endothelial cell antigens, and angiotensin II type 1 receptors, are responsible for ABMR [Colvin & Smith, cited above]. A complicated process mediates the development of antibodies upon exposure to antigens. Antigens are presented by either donor or recipient APCs to CD4+T cells (i.e., T helper cells), which then activate B cells via cytokines and costimulatory factors. Immature B cells are differentiated into either memory B cells or antibody-forming plasma cells. Plasma cells subsequently produce antibodies for longer than a year without help from T cells [Shapiro-Shelef & Calame (2005) Nat Rev Immunol 3, 230-242]. Allograft cells are not destroyed by antibodies themselves, but rather via the activation of the complement system or cytotoxic cells. Therefore, the production of DSAs does not necessarily mean that a kidney transplant recipient will experience ABMR. Complement activation plays a major role in ABMR, resulting in tissue injury and thrombosis. Complement molecules (particularly C1q) bind to the antigen-antibody complex on the graft endothelium. This interaction activates a process known as the “complement dependent cascade”, a complex process that occurs along the cellular membrane of a target cell (e.g., allograft endothelium and microvasculature). The presence of C4d on an allograft is evidence of complement activation. In fewer cases, antibodies can cause endothelial injury by a complement-independent mechanism via antibody-dependent cell-mediated cytotoxicity. This contributes to allograft injury through natural killer cells and macrophages, and it may be more related to chronic ABMR.

Previous exposure to foreign HLAs may predispose a kidney transplant recipient to an increased risk of ABMR. Patients are at risk of developing anti-HLA antibodies after solid organ transplant, blood infusion, pregnancy, and infection. Those with a significant level of anti-HLA antibodies prior to transplantation are referred to as “sensitized,” and they are at a high risk of developing posttransplant ABMR. A calculated panel reactive antibody (cPRA) is used to identify sensitized patients prior to transplant. The cPRA estimates the probability of incompatible donors for a specific recipient based on the presence of anti-HLA antibodies pretransplant. The higher the cPRA, the more sensitized the patient is, and the less likely he or she will be offered an organ. Additionally, patients with a high cPRA are more likely to develop ABMR posttransplant compared with patients who have low cPRA. In fact, patients who developed acute ABMR had a high median pretransplant peak cPRA compared with those who did not experience ABMR. Of note, although some sensitized patients may undergo desensitization protocol pretransplant, they still remain more vulnerable to developing ABMR [Kim et al. (2014) Pharmacotherapy. 34, 733-44].

Hyperacute ABMR is caused by a high presence of DSAs in a recipient at the time of transplantation. The diagnosis of hyperacute rejection typically relies on the timing of rejection, which occurs within minutes to hours after cross-clamps are released and the allograft is reperfused with blood. The allograft experiences severe cortical necrosis and thrombosis in the microvasculature, and in most cases, the allograft must be removed to avoid complications related to such a profound immunologic response. However, the incidence of hyperacute rejection in current practice is extremely low because of ABO antigen verification of donor and recipient and improved tissue typing methods conducted prior to transplant [Williams et al. (1968) N Engl J Med 12, 611-618; Racusen & Haas (2006) Clin J Am Soc Nephrol 3, 415-420].

Acute ABMR is mediated by either DSAs that were present pretransplant or de novo DSAs that developed posttransplant. Early acute ABMR is usually seen days to weeks after transplantation, but acute ABMR can occur any time posttransplant. One study reported a case of late acute ABMR that occurred 17 years posttransplant [Halloran et al. (1990) Transplantation 1, 85-91]. Histologic findings in acute ABMR are similar to hyperacute rejection, but the severity of rejection is lower. Late acute ABMR seems to be frequently accompanied by cellular rejection features [Racusen & Haas (2006) M. Clin J Am Soc Nephrol 3, 415-420]. Studies have reported that ˜5-7% of all kidney transplant recipients develop acute ABMR [Takemoto et al. (2004) Am J Transplant 7, 1033-1041]. However, the reported incidences of ABMR vary depending on factors such as the proportion of patients with preformed DSAs, detection methods and interpretation of pathohistologic findings, and the immunosuppression protocol utilized. Among sensitized patients, the incidence of acute ABMR is as high as 55% [Burns et al. (2008) Am J Transplant 12, 2684-2694]. ABMR constitutes about a fifth to half of acute rejection cases, and it has a worse prognosis than cellular rejection [Colvin & Smith, cited above].

Chronic ABMR develops slowly over months to years, and it is one of the important causes of chronic graft dysfunction [Colvin & Smith, cited above]. Chronic ABMR often causes irreversible allograft damage with a low graft survival rate and should not be confused with acute ABMR that occurs late posttransplant. In chronic ABMR, DSAs that do not lead to acute ABMR slowly activate the complement system and eventually cause histologic changes to the allograft that are distinguishable from acute ABMR and allograft dysfunction. The incidence of chronic ABMR is not known, but 60% of patients with late graft failure were found to have de novo DSAs months to years before their graft failure. In addition, concurrent cellular rejection is not uncommon in chronic ABMR [Colvin & Smith, cited above; Kim et al. cited above].

The underlying molecular mechanisms of ABMR were intensively studied over the past decade to identify potential therapeutic targets. Microvascular inflammation in general, and monocyte/macrophage infiltration in particular, have been associated with ABMR [Dean et al. (2012) Am J Transplant. 121551-1563; Fahim et al. (2007) Am J Transplant. 7, 385-393; Gibson et al. (2008) Am J Transplant. 8, 819-825; Cosio et al. (2010) Transplantation 89, 1088-1094; Sis et al. (2012) Am J Transplant. 12, 1168-1179]. Other inflammatory cells such as plasma cells, B cells, and mast cells were shown to be mostly associated with inflammatory and fibrotic changes but were not discriminatory for ABMR or TCMR [Halloran et al. (2010) Am J Transplant. 10, 2215-2222]. The significance of natural killer (NK) cells in ABMR has been recently highlighted, largely through their capacity in antibody-dependent cellular cytotoxicity (ADCC) [Resch et al. (2015) Transplantation. 99, 1335-1340]. Endothelial injury has been also consistently linked to ABMR, and evaluation of endothelial transcripts expression was proposed as an indicator of active ABMR in the latest updates of the Banff classification for renal allograft pathology [Drachenberg & Papadimitriou (2013) Transplantation. 95, 1073-1083; Sis et al. (2009) Am J Transplant. 9, 2312-2323; Loupy et al. (2017) Am J Transplant. 17, 28-41]. Also, complement activation has received extra attention in the diagnosis, prevention, and treatment of ABMR, particularly in (hyper-)acute rejection. Nevertheless, the pathophysiology of chronic ABMR may be not fully explained by complement activation [Akiyoshi et al. (2012) Hum Immunol. 73, 1226-1232]. Given the importance of HLA antibodies, B-cell inhibition (e.g. by B-cell depleting rituximab treatment) or plasma cell inactivation (by proteasome inhibition using bortezomib) have been tested in clinical studies. However, these therapies had only limited success in the prevention or treatment of ABMR [Sandal & Zand (2015) Front Biosci (Landmark Ed). 220, 743-762]. Also, complement inhibition by e.g. eculizumab is being tested in ABMR, but pilot data suggest that terminal complement inhibition is only effective in a minority of ABMR cases [Kulkarni et al. (2017) Am J Transplant. 17, 682-691].

Changes suspicious of antibody-mediated rejection reflects the phenotype of cases that have histological lesions or clinical features compatible with ABMR but not fulfilling the Banff criteria for full diagnosis of antibody-mediated rejection.

Many biopsies in patients with DSA show features of ABMR, but do not fulfill the complete histological criteria for ABMR, and are therefore categorized as “suspicious for ABMR”. In the Banff 2015 classification, all 3 features of ABMR (acute tissue injury, current antibody interaction and serologic evidence of DSA) needed to be present for final diagnosis of ABMR. However, in clinical practice, many cases do not fulfil all 3 criteria with an incomplete phenotype, and were thus classified as “suspicious for ABMR” (Banff 2015 [Loupy et al. (2015) cited above]). One category are patients meeting the histological criteria for ABMR but without detectable DSA. The latter category of “antibody-negative histology of ABMR” (DSAnegABMRh) could be explained by the presence of injurious antibodies that remain undetected by current testing methods that miss non-HLA antibodies (Banff 2013). The Banff 2017 discussions sought for a solution for the DSAnegABMRh cases and proposed to consider C4d positivity as an alternative for the DSA criterion [Haas et al. (2015) cited above]. Other cases that are suspicious of ABMR have DSA and histological lesions suggestive of ABMR, but do no reach the full histological Banff criteria of ABMR.

“biological sample” refers to any sample taken from a subject, such as a serum sample, a plasma sample, a urine sample, a blood sample, in particular a peripheral blood sample, a lymph sample, or a biopsy. In typical embodiments, the sample is a peripheral blood sample.

Solid transplant typically refers to a kidney transplant. In other embodiments the transplanted organ can be heart, lung, liver, pancreas, or small bowel.

“expression profile” refers to the expression levels of a group of genes.

“reference expression profile” refers to a profile as obtained from a healthy subject with an solid organ transplant (such as kidney) who has been diagnosed as not having or not being at risk of developing a transplant rejection.

“housekeeping gene” refers to a gene that are constitutively expressed at a relatively constant level across many or all known conditions, because they code for proteins that are constantly required by the cell, hence, they are essential to a cell and always present under any conditions. It is assumed that their expression is unaffected by experimental conditions. The proteins they code are generally involved in the basic functions necessary for the sustenance or maintenance of the cell. Non-limiting examples of housekeeping genes include HPRT1, ubiquitin C, YWHAZ, B2M, GAPDH, FPGS, DECR1, PPIB, ACTB, PSMB2, GPS1, CANX, NACA, TAX1 BP1 and PSMD2.

“probes” or “set of probes” relates to oligonucleotides binding specifically to mRNA or cDNA of a target gene. Embodiments are a single probes on a micro-array binding to mRNA or cDNA, as illustrated in the below examples. Other embodiments are pairs of primers for PCR, or double pairs of primers for nested PCR. PCR using a pair of primers and an internal primer is used in e.g. Taqman PCR as illustrated in the examples. Primers can be in solution or suspension or coupled to a substrate. Primers are optionally labelled for example with a fluorescent label, magnetic label or radioactive label.

In this multicentre, multiphase study an 8-gene expression assay in peripheral blood samples was built with good diagnostic accuracy for non-invasive diagnosis of antibody-mediated rejection. This multicentre study is a pioneer in the field of biomarker discovery and development in renal transplantation in several aspects. First, its multiphase study design with independent discovery, derivation and validation sets allowed for robust development and validation in a representative population with real-life prevalence of antibody-mediated rejection. Second, central pathology was used, minimizing the interobserver variability in the current golden standard for diagnosis of rejection and reference standard for performance of the biomarker. Third, the comparison with routine clinical markers and assessment of the net benefit of using this 8-gene assay indicate the clinical usefulness of this marker. The net benefit of the 8-gene assay for clinical decision-making is fully confirmed by the decision analysis curves. In addition, the performance of this biomarker in was assessed in different clinical scenarios. The clinical value of a biomarker in renal transplantation depends on the setting in which biopsies are performed. The high negative predictive value of the 8-gene assay in all settings is of importance and can be used to rule out antibody-mediated rejection. In addition, high sensitivity for antibody-mediated rejection, both at time of graft dysfunction and at time of stable graft function, can be of clinical use, as too many cases of antibody-mediated rejection are still missed with current clinical practice. Part of the relevance of this biomarker indeed lies in its performance independent of graft functional parameters (estimated glomerular filtration rate and proteinuria) as subclinical histological changes of antibody-mediated rejection often remain undetected but are nevertheless associated with an increased risk of graft failure [Loupy et al. (2015) J Am Soc Nephrol 26, 1721-1731]. Also the excellent diagnostic performance of the marker in the first year after transplantation is of clinical relevance, as therapeutic implications will be greatest when antibody-mediated rejection is detected early, before chronic damage has developed and the disease is more reversible [Djamali et al. (2014) Am J Transplant 4, 255-271; Loupy & Lefaucheur (2018) N Engl J Med 379, 1150-1160; Fehr & Gaspert (2012) Transpl Int. 25, 623-632]. In centers that are currently not performing protocol biopsies to detect subclinical rejection, it could be considered to include this biomarker in the follow-up of patients at increased risk of antibody-mediated rejection (e.g. patients with donor-specific antibodies), and restrict performing protocol biopsies only to patients at risk with a high value of the 8-gene assay, when antibody-mediated rejection is not excluded.

Presence of donor-specific antibodies is a well-established risk factor for antibody-mediated rejection but is a poor predictor. This is also illustrated in the validation cohort, where the presence of donor-specific antibodies had only poor diagnostic accuracy for antibody-mediated rejection. The moderate diagnostic performance of proteinuria [Naesens et al. (2016) J Am Soc Nephrol 27, 281-292], another readily available biomarker in clinical practice, was confirmed in the validation cohort. Adding the 8-gene assay to a clinical model (encompassing the presence of donor-specific antibodies, estimated glomerular filtration rate and proteinuria), increased the diagnostic accuracy to 87.8%. Given the inherent difficulties with histological diagnosis of antibody-mediated rejection as gold standard for diagnosis of antibody-mediated rejection (reproducibility, sampling error), better diagnostic accuracy of any test cannot be expected.

In conclusion, a novel 8-gene biomarker is presented with robust performance for non-invasive diagnosis of antibody-mediated rejection after kidney transplantation.

RNA levels can be determined by appropriate methods such as nucleic acid probe microarrays, Northern blots, RNase protection assays (RPA), quantitative reverse-transcription PCR (RT-PCR), dot blot assays and in-situ hybridization as disclosed in detail in EP2633078.

As exemplified in the examples section, expression levels of genes is quantitated using a real time reverse-transcription PCR (real time RT-PCR) method using the TaqMan® method.

The probe used in real time PCR assays is typically a short (ca. 20-25 bases) polynucleotide labelled with two different fluorescent dyes, i.e., a reporter dye at the 5′-terminus of the probe and a quenching dye at the 3′-terminus, although the dyes can be attached at other locations on the probe as well. For measuring a specific transcript, the probe is designed to have at least substantial sequence complementarity with a probe binding site on the specific transcript. Upstream and downstream PCR primers that bind to regions that flank the specific transcript are also added to the reaction mixture for use in amplifying the nucleic acid. When the probe is intact, energy transfer between the two fluorophores occurs and the quencher quenches emission from the reporter. During the extension phase of PCR, the probe is cleaved by the 5′-nuclease activity of a nucleic acid polymerase such as Taq polymerase, thereby releasing the reporter dye from the polynucleotide-quencher complex and resulting in an increase of reporter emission intensity that can be measured by an appropriate detection system. The fluorescence emissions created during the fluorogenic assay is measured by commercially available detectors that comprise computer software capable of recording the fluorescence intensity of reporter and quencher over the course of the amplification. These recorded values can then be used to calculate the increase in normalized reporter emission intensity on a continuous basis and ultimately quantify the amount of the mRNA being amplified.

Diagnostic accuracy of any diagnostic procedure or a test gives us an answer to the following question: “How well this test discriminates between certain two conditions of interest?”. This discriminative ability can be quantified by the measures of diagnostic accuracy:
sensitivity and specificity
positive and negative predicative values (PPV, NPV)
the area under the ROC curve (AUC)

Different measures of diagnostic accuracy relate to the different aspects of diagnostic procedure. It should be noted that measures of a test performance are not fixed indicators of a test quality and performance. Measures of diagnostic accuracy are sensitive to the characteristics of the population in which the test accuracy is evaluated. PPV and NPV largely depend on the disease prevalence, while this is not the case for sensitivity and specificity.

Application of a diagnostic test for a medical condition can yield the following combinations:
true positive (TP)—subjects with the disease with the test above the cut-off
false positive (FP)—subjects without the disease with the test above the cut-off
true negative (TN)—subjects without the disease with the test below the cut-off
false negative (FN)—subjects with the disease with the test below the cut-off

### Sensitivity

Sensitivity refers to the test's ability to correctly detect ill patients who do have the condition. Sensitivity is expressed in percentage and defines the proportion of true positive subjects with the disease in a total group of subjects with the disease (True Positive/True Positive+False Negative). Actually, sensitivity is defined as the probability of getting a positive test result in subjects with the disease. Hence, it relates to the potential of a test to recognize subjects with the disease. A negative result in a test with high sensitivity is useful for ruling out disease, as is the case with the proposed 8-gene biomarker. A high sensitivity test is reliable when its result is negative, since it rarely misdiagnoses those who have the disease. In contrast, a positive result in a test with high sensitivity is not useful for ruling in disease.

### Specificity

Specificity relates to the test's ability to correctly reject healthy patients without a condition. Specificity of a test is the proportion of healthy patients known not to have the disease, who will test negative for it. A positive result in a test with high specificity is useful for ruling in disease. A positive result signifies a high probability of the presence of disease. Specificity is defined as the proportion of subjects without the disease with negative test result in total of subjects without disease (True Negative/True Negative+False Positive).

### Positive and Negative Predictive Value

Positive predictive value (PPV) defines the probability of having the state/disease of interest in a subject with positive result. Therefore PPV represents a proportion of patients with positive test result in total of subjects with positive result (True Positive/True Positive+False Positive). Negative predictive value (NPV) describes the probability of not having a disease in a subject with a negative test result. NPV is defined as a proportion of subjects without the disease with a negative test result in total of subjects with negative test results (True Negative/True Negative+False Negative). Unlike sensitivity and specificity, predictive values are largely dependent on disease prevalence in examined population. Therefore, predictive values from one study cannot be transferred to some other setting with a different prevalence of the disease in the population. Prevalence affects PPV and NPV differently.

### ROC Curve

There is a pair of diagnostic sensitivity and specificity values for every individual threshold. To construct a Receiver Operating Characteristic (ROC) graph, these pairs of values are plotted on the graph with the 1-specificity on the x-axis and sensitivity on the y-axis. The shape of a ROC curve and the area under the curve (AUC) helps us estimate the discriminative power of a test. The closer the curve is located to upper-left hand corner and the larger the area under the curve, the better the test is at discriminating between diseased and non-diseased. The area under the curve can have any value between 0 and 1 and it is a good indicator of the goodness of the test. A perfect diagnostic test has an AUC 1.0, whereas a non-discriminating test has an area 0.5.

In the present invention, the expression level of genes in a set of genes in a body sample is subjected to a statistical analysis, and the outcome of this process is a probability value ranging between 0 and 1, which is then used for determining, under the sensitivity and specificity limitations of the particular method used, whether said individual is at risk of developing graft rejection other than T cell mediated graft rejection. The decision whether the tested individual is positive or negative is made after comparing the probability value obtained with a predetermined cut-off probability value, herein also termed “cut-off value”, ranging between 0 and 1 and preferably representing the optimal combination of sensitivity and specificity. Further to the optimal combination of sensitivity and specificity as deduced from the statistical analysis used, the cut-off value, can be subject to further parameters such as to a certain extent, is arbitrary and may be determined based, inter alia, on considerations other than optimal sensitivity and specificity, such as clinical parameters determined in other assays.

The comparison of a tested subject expression profile with said reference expression profiles, which permits prediction of the tested subject's clinical response based on his/her expression profile, can be done by those skilled in the art using statistical models or machine learning technologies as explained in EP2668287. The PLS (Partial Least Square) regression is particularly relevant to give prediction in the case of small reference samples. The comparison may also be performed using Support Vector Machines (SVM), linear regression or derivatives thereof (such as the generalized linear model abbreviated as GLM, including logistic regression), Linear Discriminant Analysis (LDA), Random Forests, k-NN (Nearest Neighbour) or PAM (Predictive Analysis of Microarrays) statistical methods. More precisely, a group of reference samples, which is generally referred to as training data, is used to select an optimal statistical algorithm that best separates responders from non-responders (like a decision rule). The best separation is usually the one that misclassifies as few samples as possible and that has the best chance to perform comparably well on a different dataset.

For example expression profile representing the normalized expression level of each one of the genes in a sample is subjected to the formula P=e^{N}/(1+e^{N}), wherein N represents the weighted sum of the natural logarithms of the normalized expression levels of said genes, with the addition of a constant; and P corresponds to the probability of developing graft rejection other than T cell mediated rejection.

Other methodologies from the field of statistics, mathematics or engineering exist, for example but not limited to decision trees, Support Vector Machines (SVM), Neural Networks and Linear Discriminant Analyses (LDA). These approaches are well known to people skilled in the art. In summary, an algorithm (which may be selected from linear regression or derivatives thereof such as generalized linear models (GLM, including logistic regression), nearest neighbour (k-NN), decision trees, support vector machines (SVM), neural networks, linear discriminant analyses (LDA), Random forests, or Predictive Analysis of Microarrays (PAM) is calibrated based on a group of reference samples and then applied to the test sample.

The invention further relates to a computer readable medium having computer readable instructions recorded thereon to perform the calculation of the expression profiles of the subjects to be tested, the comparison with a reference expression profile and the probability that the subject is at risk of developing graft rejection other than T cell mediated graft rejection of the transplanted organ. Embodiments of such computer readable media are described in EP2668287.

### EXAMPLES

### Example 1 Study Design, Patient Population and Sample Collection

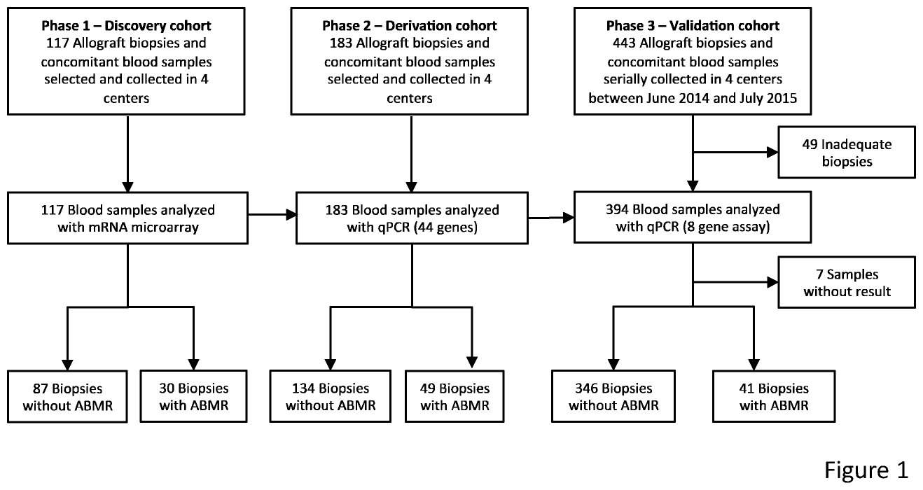

In a multicentre study, 687 peripheral blood samples obtained at the time of a renal allograft biopsy were evaluated, 120 with antibody-mediated rejection and 567 without (FIG. 1). Protocol renal allograft biopsies were performed at 3, 12, and sometimes at 24 months after transplantation, according to local centre practice, in addition to clinically indicated biopsies (biopsies at time of graft dysfunction). All adult patients who had received a single kidney transplant at these institutions and who provided written informed consent, were eligible. Recipients of combined transplantations were excluded. All transplantations were performed with negative complement-dependent cytotoxicity cross-matches. Institutional review boards and national regulatory agencies (when required) approved the study protocol at each clinical centre.

Baseline Characteristics Patients' demographics and clinical characteristics of the three independent peripheral blood sample sets are provided in Table 1.

TABLE 1
Characteristics of the patients and biopsies included in the study (N = 694).^{&}

| | | | |
|---|---|---|---|
| | Discovery Phase | Derivation | Validation Phase |
| | (N = 117) | Phase (N = 183) | (N = 387) |
| | Mean (median) ± | Mean (median) ± | Mean (median) ± |
| | standard | standard | standard |
| | deviation (min-max) | deviation (min-max) | deviation (min-max) |
| Variable | or no. (%) | or no. (%) | or no. (%) |

Transplant characteristics

| Recipient age at | 49.2 (51.8) ± 13.8 | | 49.2 (51.7) ± 15.1 | | 50.0 (51.6) ± 15.3 | |
|---|---|---|---|---|---|---|
| transplantation | (18.8-75.3) | | (14.8-74.0) | | (2.72-78.5) | |
| (years) | | | | | | |
| Recipient age at | 52.0 (53.3) ± 13.2 | | 52.0 (54.2) ± 14.8 | | 52.4 (54.1) ± 14.4 | |
| time of biopsy | (19.9-76.3) | | (20.3-77.6) | | (19.0-79.6) | |
| (years) | | | | | | |
| Recipient gender | 69/48 | | 100/83 | | 240/147 | |
| (male/female) | (59.0%/41.0%) | | (54.6%/45.4%) | | (62.0%/38.0%) | |
| Repeat | 20 | (17.1%) | 46 | (25.1%) | 69 | (17.8%) |
| transplantation | | | | | | |
| Recipient ethnicity | 97/6/5/9 | | 161/2/4/12 | | 338/3/6/37* | |
| (European/Asian/ | (82.9%/5.13%/4.27%/7.69%) | | (88%/1.1%/2.2%/6.6%) | | (87.3%/0.8%/1.6%/9.6%) | |
| African/Other) | | | | | | |
| Donor age (years) | 49.8 (51.0) ± 15.8 | | 49.5 (52.0) ± 16.3 | | 50.3 (52.0) ± 15.5 | |
| | (13.0-88.0) | | (8.0-89.0) | | (5.0-91.0) | |
| Donor gender | 54/59* | | 81/87* | | 192/187* | |
| (male/female) | (47.8%/52.2%) | | (44.3%/47.5%) | | (49.6%/48.3%) | |
| Deceased/Living | 96/21 | | 150/32* | | 295/88* | |
| donor | (82.1%/18.0%) | | (82%/17.5%) | | (76.2%/22.7%) | |
| Heart-beating/ | 91/5 | | 132/18* | | 263/32* | |
| Non-heart-beating | (94.8%/5.21%) | | (72.1%/9.8%) | | (68.0%/8.3%) | |
| donor | | | | | | |
| Cold ischemia time | 13.8 (14.2) ± 8.25 | | 13.1 (12.8) ± 7.6 | | 12.1* (12.7) ± 7.9 | |
| (hours) | (0.28-38.2) | | (0.43-36.0) | | (0.27-35.8) | |
| Primary immune- | | | | | | |
| suppression | | | | | | |
| Cyclosporine | 22 | (18.9%) | 33 | (18.0%) | 54 | (14.0%) |
| Tacrolimus | 95 | (81.2%) | 143 | (78.1%) | 327 | (84.5%) |
| Mycophenolate | 112 | (95.7%) | 162 | (88.5%) | 328 | (84.8%) |
| mTOR inhibitor | 1 | (0.85%) | 7 | (3.8%) | 46 | (11.9%) |
| Azathioprine | 1 | (0.85%) | 1 | (0.55%) | 9 | (2.3%) |
| Corticosteroids | 117 | (100%) | 178 | (97.3%) | 381 | (98.5%) |
| Additional | 88/116* | (75.9%) | 112/178* | (61.2%) | 307* | (79.3%) |
| induction therapy | | | | | | |

Biopsy characteristics

| Indication/ protocol | 47/70 | | 71/112 | | 134/253 | |
|---|---|---|---|---|---|---|
| biopsy | (40.2%/59.8%) | | (38.8%/61.2%) | | (34.6%/65.4%) | |
| Time after | 986 (365) ± 1497 | | 933 (361) ± 1665 | | 908 (359) ± 1733 | |
| transplantation | (6-7992) | | (4-10063) | | (6-12564) | |
| (days) | | | | | | |
| Biopsy time after | | | | | | |
| transplantation | | | | | | |
| <1 year | 59 | (50.4%) | 97 | (53.0%) | 207 | (53.5%) |
| >1 year | 58 | (49.6%) | 86 | (47%) | 180 | (46.5%) |
| MDRD eGFR, | 42.7 (39.3) ± 19.6 | | 41.3 (40.0) ± 21.2 | | 43.0 (41.8) ± 17.5 | |
| (ml/min/1.73 m²)^{‡} | (5.77-111) | | (5.30-101.2) | | (5.75-96.2) | |
| Proteinuria (g/g | 0.53 (0.18) ± 0.90 | | 0.58 (0.16) ± 1.09 | | 0.43* (0.14) ± 0.96 | |
| creatinine)§ | (0.02-4.59) | | (0.03-6.64) | | (0.00-8.04) | |
| Immunosuppression at time of biopsy | | | | | | |
| Cyclosporine | 13 | (11.1%) | 16 | (8.7%) | 40 | (10.3%) |
| Tacrolimus | 95 | (81.2%) | 152 | (83.1%) | 331 | (85.5%) |
| Mycophenolate | 99 | (84.6%) | 163 | (89.1%) | 320 | (82.7%) |
| Azathioprine | 6 | (5.13%) | 5 | (2.73%) | 9 | (2.3%) |
| mTOR inhibitor | 4 | (3.42%) | 10 | (5.5%) | 49 | (12.7%) |
| Corticosteroids | 101 | (86.3%) | 166 | (90.7%) | 352 | (91.0%) |
| Histological | | | | | | |
| diagnosis | | | | | | |
| No rejection | 73 | (62.4%) | 104 | (56.8%) | 330 | (85.3%) |
| T-cell mediated | | | | | | |
| rejection^{†} | | | | | | |
| No | 91 | (77.8%) | 124 | (67.8%) | 368 | (95.1%) |
| Borderline changes | 19 | (16.2%) | 36 | (19.7%) | 15 | (3.9%) |
| Grade 1 or 2 | 7 | (5.98%) | 23 | (12.6%) | 4 | (1.0%) |
| Antibody-mediated | 30 | (25.6%) | 49 | (26.8%) | 41 | (10.6%) |
| rejection^{†} | | | | | | |
| Mixed rejection | 9 | (7,69%) | 29 | (15.9%) | 3 | (0.8%) |
| Interstitial fibrosis/tubular atrophy | | | | | | |
| Grade 0 | 50 | (42.7%) | 104 | (56.8%) | 182 | (47.0%) |
| Grade 1 | 30 | (25.6%) | 26 | (14.2%) | 93 | (24.0%) |
| Grade 2-3 | 37 | (31.6%) | 52 | (28.4%) | 112 | (28.9%) |

| | | | | |
|---|---|---|---|---|
| Polyomavirus- | — | — | 14 | (3.6%) |
| associated | | | | |
| nephropathy | | | | |
| De novo/recurrent | — | — | 26 | (6.7%) |
| glomerulonephritis | | | | |

*Missing data on donor gender and donor type.
^{†}The groups with antibody-mediated rejection and T-cell mediated rejection contain also mixed cases.
^{‡}Similar eGFR (mL/min/1.73 m2) for presence vs. absence of antibody-mediated rejection in the discovery cohort (38.7 ± 16.9 vs. 44.0 ± 20.3, p = 0.20), significantly lower eGFR antibody-mediated rejection patients in the derivation and validation cohorts (31.9 ± 18.7 vs. 44.8 ± 21.1, p <0.001 and 34.1 ± 21.4 vs. 44.0 ± 16.7, p = 0.006, respectively).
§Proteinuria (g/g creatinine) was significantly higher for patients with versus without antibody-mediated rejection in all three phases (1.02 ± 1.19 vs. 0.37 ± 0.71, p = <0.001; 1.40 ± 1.73 vs 0.29 ± 0.48, p < 0.001; 1.40 ± 1.78 vs. 0.31 ± 0.69, p <0.001, in the discovery, derivation and validation phase, respectively).

Details on the clinical characteristics of the biopsy samples used for micro-array gene expression (N=95) were provided separately [Yazdan et al. (2019) Kindney Int. 95, 188-198].

Two needle cores were taken at each kidney allograft biopsy. One was used for histology, at least half of the other one was immediately stored in Allprotect Tissue Reagent® (Qiagen Benelux BV, Venlo, The Netherlands) for RNA expression analysis. All biopsies included in this study were read by local pathologists and then reviewed and graded in a blinded fashion by a central pathologist independent from the original center. All biopsies were rescored semiquantitatively according to the updated Banff 2017 classification. In case of discrepancy in the final diagnosis between local and central pathology, a second central pathologist reviewed the biopsy, and a final diagnosis was agreed between both pathologists. In the discovery and derivation case-control phases of the study, samples were included only if there was concordance in the final diagnosis between local and central pathology. For the validation phase of the study, only central pathology diagnoses were used for correlation with the peripheral blood gene expression analysis. Local pathology reading was not taken into account at this stage. The presence or absence of donor-specific HLA antibodies was assessed routinely in all patients, per centre practice.

TABLE 2
Histological characteristics of the biopsies included
in the 3 × BIOS2 study, according to study phase.

| | Discovery | | Derivation | | Validation | |
|---|---|---|---|---|---|---|
| Histological | cohort | | cohort | | cohort | |
| parameter | (N = 117) | | (N = 183) | | (N = 387) | |
| Glomerulitis >0 | 28 | (23.9%) | 42 | (23%) | 47 | (12.1%) |
| Peritubular | 33 | (28.2%) | 41 | (22.4%) | 62 | (16.0%) |
| capillaritis >0 | | | | | | |
| Microcirculation | 29 | (24.8%) | 35 | (19.1%) | 43 | (11.1%) |
| inflammation >1 | | | | | | |
| Transplant | 14 | (12.0%) | 14 | (7.6%) | 29 | (7.5%) |
| glomerulopathy >0 | | | | | | |
| C4d deposition | 16 | (13.7%) | 38 | (20.8%) | 77 | (19.9%) |
| Interstitial | 26 | (22.2%) | 32 | (17.5%) | 23 | (5.9%) |
| inflammation >0 | | | | | | |
| Tubulitis >0 | 32 | (27.4%) | 65 | (35.5%) | 47 | (12.1%) |
| Total i score >0 | 48 | (41.0%) | 59 | (32.2%) | 69 | (17.8%) |
| Intimal arteritis >0 | 4 | (3.42%) | 19 | (10.4%) | 6 | (1.55%) |
| Tubular atrophy >1 | 39 | (31.6%) | 49 | (26.8%) | 113 | (29.2%) |
| Interstitial fibrosis >1 | 38 | (32.5%) | 53 | (29%) | 115 | (29.7%) |
| Arteriolar hyalinosis >1 | 32 | (27.4%) | 45 | (24.6%) | 82* | (21.2%) |
| Vascular intimal | 29 | (24.8%) | 58 | (31.7%) | 125* | (32.3%) |
| thickening >1 | | | | | | |
| DSA positivity | 32 | (27.4%) | 94 | (51.4%) | 64 | (16.5%) |

In the discovery set, 117 blood samples and 95 biopsy samples were used for genome-wide expression analysis. Samples were selected based on availability and histological criteria of concomitant renal allograft biopsies (excluding cases with diagnosis of glomerulonephritis or polyomavirus nephropathy, and cases with unclear diagnosis), while graft function was not taken into account. The same study design was used for the derivation cohort (N=183), for targeted validation of the results obtained in the discovery set, and derivation of the multigene marker. In the validation cohort, all samples with concomitant adequate renal allograft biopsy histology, prospectively collected between Jun. 24, 2014 and Jul. 2, 2015 were serially included without selection on histology, demographics or time. The gene expression profile was not complete in seven of these samples, leading to a total of 387 cases in the validation phase.

### Example 2 Primary and Secondary End Points

The primary end point was the diagnostic accuracy of a multigene marker for antibody-mediated rejection in the validation cohort. Secondary endpoints were the diagnostic accuracy in specific clinical situations (at time of graft dysfunction versus at time of stable graft function, early versus later after transplantation), comparison with traditional markers used in kidney transplantation (proteinuria and estimated glomerular filtration rate) and net benefit for clinical decision-making.

### Example 3 Sample Collection and Biopsy Scoring

Peripheral blood samples were collected at time of the renal allograft biopsies, directly in PAXgene Blood RNA Tubes® (Qiagen Benelux BV, Venlo, The Netherlands). Two needle cores were taken at each kidney allograft biopsy. One was used for histology, at least half of the other one was immediately stored in Allprotect Tissue Reagent® (Qiagen Benelux BV, Venlo, The Netherlands) for RNA expression analysis (in the discovery set). All biopsies included in this study were reviewed and graded in a blinded fashion by a central pathologist independent from the original center. All biopsies were rescored semiquantitatively according to the updated Banff 2017 classification [Haas et al. (2018) Am J Transplant 18, 293-307].

In the discovery cohort, RNA extracted from blood and biopsies was hybridized onto Affymetrix GeneChip Human Genome U133 Plus 2.0 Arrays (Affymetrix Inc., High Wycombe HP10 0HH, UK). In the derivation and validation cohorts, RNA expression analysis of mRNA extracted from blood samples was evaluated by real-time polymerase chain reaction (RT-PCR) using OpenArray® technology on the Quantstudio™ 12K Flex Real-Time PCR System (Life Technologies Europe BV, Ghent, Belgium) with ACTB, GAPDH and SDHA as endogenous controls. Peripheral blood samples were collected at time of the renal allograft biopsies, directly in PAXgene Blood RNA Tubes® (Qiagen Benelux BV, Venlo, The Netherlands). The Paxgene tubes were stored at ambient temperature for at least 24 hours, and then stored at −80° C. until extraction. Total RNA was extracted using the PAXgene Blood miRNA Kit® (Qiagen SA, Courtaboeuf, France). The yield and purity of RNA was measured using a NanoDrop® ND-1000 spectrophotometer (Thermo Scientific™, Life Technologies Europe BV, Ghent, Belgium). In the discovery cohort, RNA integrity was assessed using the RNA 6000 Nano LabChip® kit (Agilent Technologies Belgium NV, Diegem, Belgium) on the Bioanalyzer 2100 Instrument™ (Agilent Technologies Belgium NV, Diegem, Belgium), and globin mRNA was depleted using the GLOBINclear™ Kit (Invitrogen™, Life Technologies Europe BV, Ghent, Belgium). After globin mRNA depletion of samples in the discovery cohort, and for all samples in the derivation and validation cohorts, the quantity (absorbance at 260 nm) and purity (ratio of the absorbance at 230, 260 and 280 nm) of the isolated RNA were measured using the NanoDrop ND-1000™ spectrophotometer (NanoDrop Technologies, Inc., Rockland, Del., USA). After extraction and quality control, the extracted RNA samples were stored at −80° C.

RNA expression analysis of mRNA extracted from blood samples of the derivation and validation cohort was evaluated by RT-PCR using OpenArray® technology, a real-time PCR-based solution for high-throughput gene expression analysis on the Quantstudio™ 12K Flex Real-Time PCR System (Life Technologies Europe BV, Ghent, Belgium). cDNA synthesis was executed according the manufacturer with 50 ng mRNA with the SuperScript® VILO™ cDNA Synthesis Kit (Life Technologies Europe, Bleiswijk, The Netherlands). The synthesized cDNA was first pre-amplified, and then mixed with TaqMan® Universal PCR Master Mix (Applied Biosystems™, Life Technologies Europe BV, Ghent, Belgium) and injected onto the OpenArray™ slides using the OpenArray® AccuFill™ System (Applied Biosystems™, Life Technologies Europe BV, Ghent, Belgium), according to the manufacturer's instructions. The OpenArray® slides were spotted with the selected TaqMan® assays including three endogenous controls ACTB, GAPDH and SDHA. These housekeeping genes were selected and tested using the geNorm algorithm in the qbase+ software (Biogazelle, Zwijnaarde, Belgium). Raw data were analysed using the QuantStudio™ 12K Flex Software (Applied Biosystems™, Life Technologies Europe BV, Ghent, Belgium). Gene expression of each target (ΔC_{q}) was calculated relative to the mean expression of three endogenous controls (ACTB, GAPDH and SDHA), using the cycle value (C_{q}) method as determined by the relative threshold cycle (Cᵣₜ) algorithm (Applied Biosystems™, Life Technologies Europe BV, Ghent, Belgium).

### Microarray Gene Expression in Peripheral Blood Samples and Biopsy Samples

In the discovery cohort, total RNA extracted from PAXgene blood tubes was hybridized onto Affymetrix GeneChip Human Genome U133 Plus 2.0 Arrays (Affymetrix Inc., High Wycombe HP10 0HH, UK), according to the manufacturer's instructions. This whole human genome expression array covers genes for analysis of 54,613 different probes. GeneChip® Scanner 3000 7G System (Affymetrix Inc., High Wycombe HP10 0HH, UK) and GeneChip® Command Console® Software (AGCC) were used for scanning the arrays and generating the images, respectively. The .CEL files were processed with RMA background correction and normalization, and log 2 scaled. Of the 131 blood RNA samples of the discovery cohort, 121 survived pre-hybridization quality control checks, of which 117 were retained after outlier elimination and filtering. These 117 blood RNA samples in the discovery cohort were used for further statistical analysis.

RNA was extracted from the renal biopsies using the Allprep DNA/RNA/miRNA Universal Kit® (Qiagen Benelux BV, Venlo, The Netherlands) on a QIAcube instrument (Qiagen Benelux BV, Venlo, The Netherlands). The quantity (absorbance at 260 nm) and purity (ratio of the absorbance at 230, 260 and 280 nm) of the RNA isolated from the biopsies were measured using the NanoDrop ND-1000™ spectrophotometer (Thermo Scientific™, Life Technologies Europe BV, Ghent, Belgium). RNA integrity was evaluated using the Eukaryote nano/pico RNA Kit® (Agilent Technologies Belgium NV, Diegem, Belgium) on the Bioanalyzer 2100 Instrument™ (Agilent Technologies Belgium NV, Diegem, Belgium). Samples were stored at −80° C. until further analysis.

RNA extracted from the biopsy samples was first amplified and biotinylated to complementary RNA (cRNA) using the GeneChip® 3′ IVT PLUS Reagent Kit (Affymetrix Inc., High Wycombe HP10 0HH, UK) and subsequently hybridized onto Affymetrix GeneChip Human Genome U133 Plus 2.0 Arrays (Affymetrix Inc., High Wycombe HP10 0HH, UK), which covers over 54k transcripts, according to the manufacturer's instructions. The arrays were scanned using the GeneChip® Scanner 3000 7G System (Affymetrix Inc., High Wycombe HP10 0HH, UK), and image files were generated using the GeneChip® Command Console® Software (AGCC). Finally, Robust Multichip Average (RMA) background correction and normalization was performed using the Affymetrix Expression Console Software, and expression values were log 2 scaled. Of the 121 biopsies that were sent to the Laboratory of Nephrology, 109 survived pre-hybridization quality control checks, and were analyzed. Outlier analysis and filtering was performed based on Hotelling's T² test on principal component analysis (PCA) components and quantile distribution of the profiles, which left 95 biopsies for further statistical analysis. Of these 95 samples, 83 samples had data on global mRNA gene expression in the peripheral blood sample that was obtained on the same day.

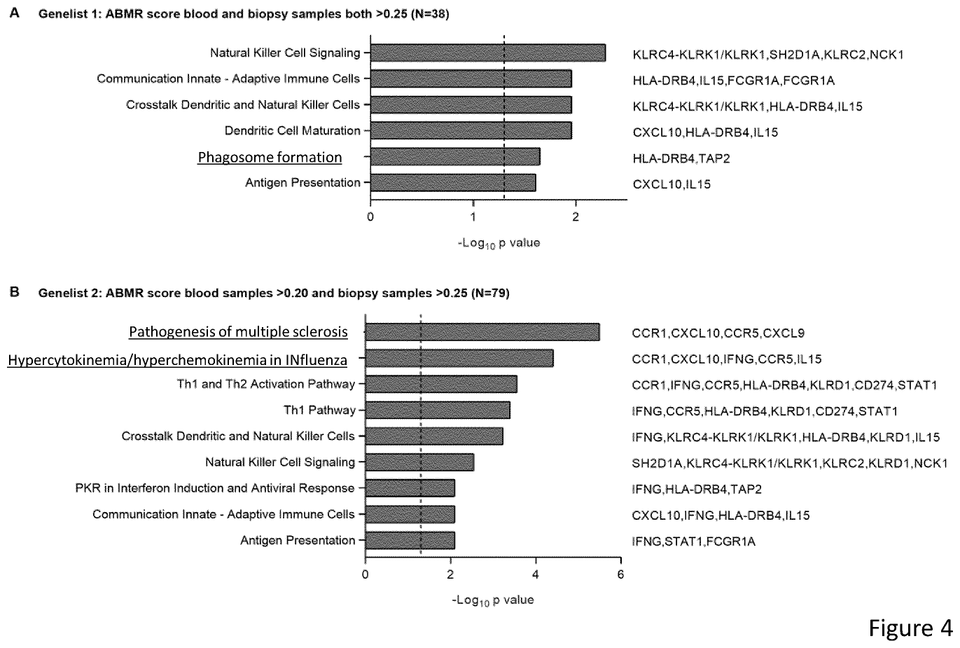

In peripheral blood and biopsy samples, respectively 970 and 783 probesets (730 and 576 individual genes) had an ABMR score >0.25. Pathway enrichment analysis of the biopsy signature was previously published [Yazdani et al. (2019) Kidney int. 95, 188-198]. Based on ABMR and TCMR scores in peripheral blood and biopsies, 2 genelists were determined FIG. 4. In both genelists, there was significant enrichment in natural killer cell signalling and antigen presentation pathways. Predicted upstream factor analysis was conducted and found interferons and interferon regulatory factors to be the most likely upstream regulators.

### Gene Signature Identification and Model Development in the Derivation Cohort

From the genelists obtained in the discovery phase, 44 transcripts were selected for RT-PCR analysis in the derivation cohort, based on combinations of ABMR and TCMR scores in blood and transplant biopsies, robustness of the results with different probesets of the same gene and by their involvement in relevant canonical pathways Table 3).

TABLE 3
Blood and biopsy scores for the 44 selected gene transcripts used
for biomarker development in the derivation cohort (N = 183).

| | | | Blood | Blood | Biopsy | Biopsy |
|---|---|---|---|---|---|---|
| Gene | Gene | | ABMR | TCMR | ABMR | TCMR |
| symbol | description | TaqMan ® assay | score | score | score | score |
| CASP4 | Caspase 4 | Hs01031951_m1 | 0.31 | 0.13 | 0.18 | 0.15 |
| CCR3 | C-C Motif | Hs00266213_s1 | 0.26 | 0.18 | 0.00 | 0.00 |
| | Chemokine | | | | | |
| | Receptor 3 | | | | | |
| CCR7 | C-C Motif | Hs01013469_m1 | 0.28 | 0.10 | 0.19 | 0.24 |
| | Chemokine | | | | | |
| | Receptor 7 | | | | | |
| CD160 | CD160 Molecule | Hs00199894_m1 | 0.20 | 0.18 | 0.41 | 0.19 |
| CD274 | CD274 Molecule | Hs01125301_m1 | 0.33 | 0.21 | 0.28 | 0.14 |
| CD38 | CD38 Molecule | Hs01120071_m1 | 0.28 | 0.10 | 0.29 | 0.17 |
| CD46 | CD46 Molecule | Hs00611257_m1 | 0.29 | 0.19 | 0.04 | 0.00 |
| CFP | Complement | Hs00175252_m1 | 0.24 | 0.10 | 0.38 | 0.19 |
| | Factor Properdin | | | | | |
| CLEC7A | C-Type Lectin | Hs01902549_s1 | 0.29 | 0.32 | 0.31 | 0.23 |
| | Domain | | | | | |
| | Containing 7A | | | | | |
| CLU | Clusterin | Hs00156548_m1 | 0.29 | 0.13 | 0.12 | 0.11 |
| CRTAM | Cytotoxic And | Hs00219699_m1 | 0.27 | 0.17 | 0.35 | 0.23 |
| | Regulatory T-Cell | | | | | |
| | Molecule | | | | | |
| CXCL10 | C-X-C Motif | Hs01124252_g1 | 0.30 | 0.10 | 0.38 | 0.25 |
| | Chemokine | | | | | |
| | Ligand 10 | | | | | |
| CXCL11 | C-X-C Motif | Hs00171138_m1 | 0.08 | 0.14 | 0.49 | 0.25 |
| | Chemokine | | | | | |
| | Ligand 11 | | | | | |
| CXCL9 | C-X-C Motif | Hs00171065_m1 | 0.20 | 0.18 | 0.45 | 0.24 |
| | Chemokine | | | | | |
| | Ligand 9 | | | | | |
| CYLD | CYLD Lysine 63 | Hs00211000_m1 | 0.30 | 0.22 | 0.16 | 0.19 |
| | Deubiquitinase | | | | | |
| ETV7 | ETS Variant 7 | Hs00903229_m1 | 0.24 | 0.22 | 0.28 | 0.19 |
| FAS | Fas Cell Surface | Hs00163653_m1 | 0.33 | 0.19 | 0.15 | 0.08 |
| | Death Receptor | | | | | |
| FCGR1A | Fragment Of IgG | Hs00417598_m1 | 0.28 | 0.26 | 0.31 | 0.23 |
| | Receptor Ia | | | | | |
| FCGR1B | Fragment Of IgG | Hs02387778_mH | 0.28 | 0.26 | 0.31 | 0.23 |
| | Receptor Ib | | | | | |
| GBP1 | Guanylate Binding | Hs00977005_m1 | 0.25 | 0.22 | 0.38 | 0.17 |
| | Protein 1 | | | | | |
| GBP2 | Guanylate Binding | Hs00894837_m1 | 0.31 | 0.14 | 0.31 | 0.23 |
| | Protein 2 | | | | | |
| GBP4 | Guanylate Binding | Hs00925073_m1 | 0.24 | 0.14 | 0.41 | 0.16 |
| | Protein 4 | | | | | |
| GBP5 | Guanylate Binding | Hs00369472_m1 | 0.31 | 0.18 | 0.35 | 0.19 |
| | Protein 5 | | | | | |
| IFIH1 | Interferon | Hs00223420_m1 | 0.33 | 0.13 | 0.21 | 0.04 |
| | Induced With | | | | | |
| | Helicase C | | | | | |
| | Domain 1 | | | | | |
| IFNG | Interferon | Hs00989291_m1 | 0.22 | 0.10 | 0.31 | 0.13 |
| | Gamma | | | | | |
| IL15 | Interleukin 15 | Hs01003716_m1 | 0.25 | 0.19 | 0.28 | 0.14 |
| ITGA2B | Integrin Subunit | Hs01116228_m1 | 0.27 | 0.13 | 0.08 | 0.00 |
| | Alpha 2b | | | | | |
| JAK2 | Janus kinase 2 | Hs01078136_m1 | 0.33 | 0.21 | 0.22 | 0.15 |
| KCNJ2 | Potassium | Hs00265315_m1 | 0.28 | 0.14 | 0.31 | 0.03 |
| | Voltage-Gated | | | | | |
| | Channel | | | | | |
| | Subfamily J | | | | | |
| | Member 2 | | | | | |
| KLRC1 | Killer Cell Lectin | Hs00970273_g1 | 0.29 | 0.25 | 0.35 | 0.20 |
| | Like Receptor C1 | | | | | |
| KLRC2 | Killer Cell Lectin | Hs02379574_g1 | 0.29 | 0.25 | 0.35 | 0.20 |
| | Like Receptor C2 | | | | | |
| KLRC4- | KLRC4-KLRK1 | Hs00183683_m1 | 0.27 | 0.14 | 0.35 | 0.23 |
| KLRK1 | Readthrough | | | | | |
| KLRD1 | Killer Cell Lectin | Hs00233844_m1 | 0.24 | 0.18 | 0.42 | 0.20 |
| | Like Receptor D1 | | | | | |
| MAP2K5 | Mitogen-Activated | Hs00177134_m1 | 0.32 | 0.10 | 0.08 | 0.04 |
| | Protein Kinase | | | | | |
| | Kinase 5 | | | | | |
| MAP3K8 | Mitogen-Activated | Hs00178297_m1 | 0.29 | 0.19 | 0.14 | 0.10 |
| | Protein Kinase | | | | | |
| | Kinase Kinase 8 | | | | | |
| MRE11A | MRE11 Homolog, | Hs00967437_m1 | 0.33 | 0.13 | 0.04 | 0.00 |
| | Double Strand | | | | | |
| | Break Repair | | | | | |
| | Nuclease | | | | | |
| SELP | Selectin P | Hs01032845_m1 | 0.27 | 0.13 | 0.12 | 0.04 |
| SH2D1A | SH2 Domain | Hs00158978_m1 | 0.27 | 0.18 | 0.28 | 0.23 |
| | Containing 1A | | | | | |
| SLAMF7 | SLAM Family | Hs00900280_m1 | 0.24 | 0.20 | 0.35 | 0.23 |
| | Member 7 | | | | | |
| STARD4 | StAR Related | Hs00287823_m1 | 0.31 | 0.25 | 0.31 | 0.03 |
| | Lipid Transfer | | | | | |
| | Domain | | | | | |
| | Containing 4 | | | | | |
| STX11 | Syntaxin 11 | Hs01891623_s1 | 0.20 | 0.10 | 0.31 | 0.19 |
| TAB2 | TGF-Beta | Hs00248373_m1 | 0.31 | 0.15 | 0.08 | 0.00 |
| | Activated Kinase | | | | | |
| | 1/MAP3K7 | | | | | |
| | Binding Protein 2 | | | | | |
| TAP2 | Transporter 2, | Hs00241060_m1 | 0.31 | 0.22 | 0.38 | 0.15 |
| | ATP Binding | | | | | |
| | Cassette | | | | | |
| | Subfamily B | | | | | |
| | Member | | | | | |
| TIMP1 | TIMP | Hs00171558_m1 | 0.31 | 0.13 | 0.11 | 0.25 |
| | Metallopeptidase | | | | | |
| | Inhibitor 1 | | | | | |

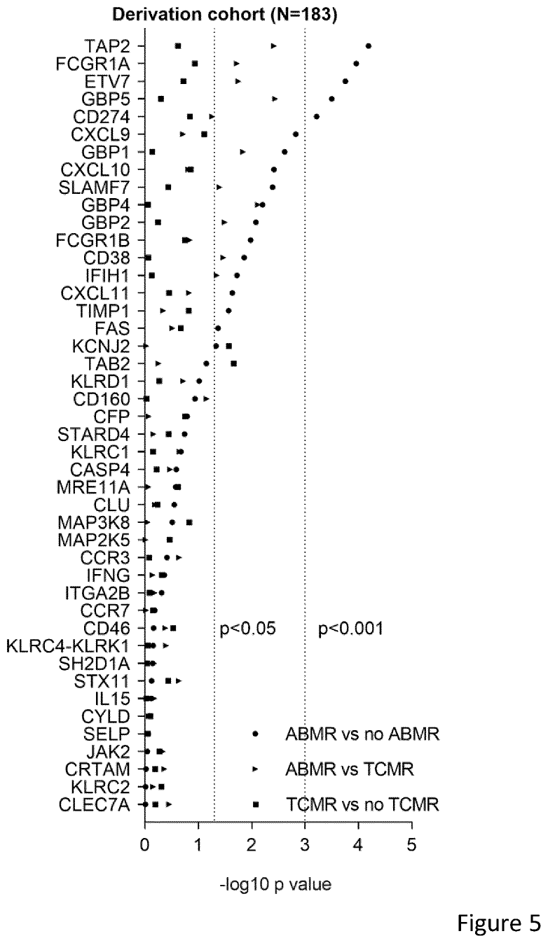

First, 26 genes with ABMR score >0.25 and TCMR score <0.20 in blood were selected (of these selected transcripts, 9 also had a high ABMR score >0.25 in kidney biopsies. Additionally 17 genes were selected with an ABMR score >0.25 in biopsies and ABMR score >0.20 in peripheral blood. Finally, given the homology with CXCL10 and ABMR score of 0.49 in biopsy samples (but only 0.08 in blood), CXCL11 was added to the gene panel in the derivation phase. The univariate associations of the expression of these 44 genes with antibody-mediated rejection are shown in FIG. 5. From these 44 genes, a gene signature specific for antibody-mediated rejection was identified on the samples of the derivation cohort (N=183), and included the following 8 genes: CXCL10, FCGR1A, FCGR1B, GBP1, GBP4, IL15, KLRC1, TIMP1 (table 4).

TABLE 4
Genes included in the 8-gene marker by PCR assay ID.

| | | Blood | Blood | Biopsy | Biopsy |
|---|---|---|---|---|---|
| Gene | | ABMR | TCMR | ABMR | TCMR |
| symbol | Gene description | score | score | score | score |
| CXCL10 | C-X-C Motif Chemokine | 0.30 | 0.10 | 0.38 | 0.25 |
| | Ligand 10 | | | | |
| FCGR1A | Fragment Of IgG Receptor Ia | 0.28 | 0.26 | 0.31 | 0.23 |
| FCGR1B | Fragment Of IgG Receptor Ib | 0.28 | 0.26 | 0.31 | 0.23 |
| GBP1 | Guanylate Binding Protein 1 | 0.25 | 0.22 | 0.38 | 0.17 |
| GBP4 | Guanylate Binding Protein 4 | 0.24 | 0.14 | 0.41 | 0.16 |
| IL15 | Interleukin 15 | 0.25 | 0.19 | 0.28 | 0.14 |
| KLRC1 | Killer Cell Lectin Like Receptor C1 | 0.29 | 0.25 | 0.35 | 0.20 |
| TIMP1 | TIMP Metallopeptidase Inhibitor 1 | 0.31 | 0.13 | 0.11 | 0.25 |

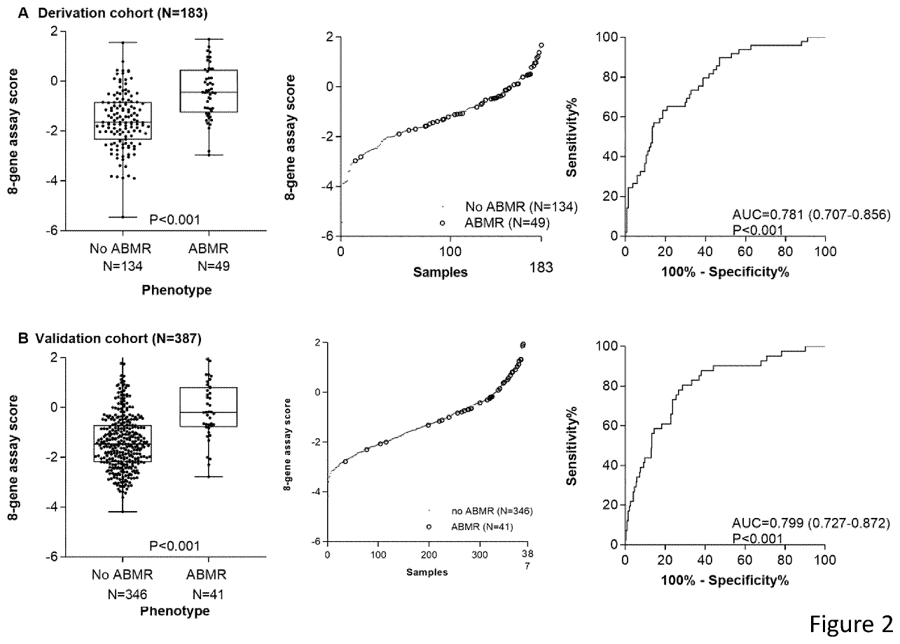

Subsequently, this 8-gene signature was used to build a logistic regression model with nested loop internal cross-validation for discrimination of cases with versus without antibody-mediated rejection in the derivation cohort. Applied to the samples of the derivation cohort, this gene signature and logistic regression model yielded an accuracy of 78.1% (95% confidence interval [CI], 70.7 to 85.6; p<0.001)(FIG. 2).

Logistic regression model for calculation of the 8-gene marker:

log(odds ABMR)=−2.52117530467713−0.246299584235795*ΔCq_CXCL10+0.086312696500064*ΔCq_GBP1+0.756045788221541*ΔCq_IL15−1.21264676000935*ΔCq_FCGR1A+0.45174226032547*ΔCq_FCGR1B−0.311501788690193*ΔCq_GBP4−0.397876959184759*ΔCq_KLRC1+1.03160242141683*ΔCq_TIMP1

ΔCq_gene corresponds to the mean delta-Cq of each gene, which is the difference between the measured Cq value of each gene and the mean Cq value of three endogenous controls ACTB, GAPDH and SDHA.

### Example 4 Data Analysis

### Discovery Phase

Given the co-occurrence of antibody-mediated and T-cell mediated rejection in several biopsies, this statistical pipeline was constructed to enable discovery of mRNA markers that were specific for antibody-mediated rejection using several class definitions: pure antibody-mediated rejection versus no rejection; pure antibody-mediated rejection versus pure T-cell mediated rejection; pure antibody-mediated rejection versus all others (pure T-cell mediated rejection, no rejection and mixed rejection), and antibody-mediated rejection (pure+mixed) versus no antibody-mediated rejection (no rejection+pure T-cell mediated rejection). Gene expression differences between these class definitions were compared using 5 different methods: Sparse Partial Least Squares (SPLS) [Chun & Keleş (2010) J R Stat Soc Series B Stat Methodol 72, 3-25], Support Vector Machine—Recursive Feature Elimination (SVM-RFE) [Guyon et al. (2002) Mach Learn 46, 389-422], Random Forrest [Breiman (2001) Mach Learn 45, 5-32], Elastic-Net [Zou & Hastie (2005) R Stat Soc Ser B 67, 301-320] and Shrunken Centroids [Tibshirani et al. (2002) Proc Natl Acad Sci 99, 6567-6572], with 10-fold cross-validation resampling. The discriminative scores of each transcript within each multivariate model were then integrated, to yield a “multivariate score” for antibody-mediated rejection for each transcript (“antibody-mediated rejection score”). The multivariate score for a given transcript was computed as S=⅕Σₘ₌₁⁵Aₘ²×σₘ where Aₘ was the accuracy of the model obtained using method m and σₘ was a Boolean value that indicates if the given transcript was selected by the method m or not. The multivariate score for a given transcript was then defined as the mean of the square accuracy of the models obtained among the set of multivariate methods that selected the transcript and reflects the number of times it was retained and involved in accurate models. A multivariate ABMR score >0.25 was used as threshold for discriminative performance. Similarly, a “T-cell mediated rejection score” was calculated using the same analytical pipeline and similar class. The combination of the antibody-mediated rejection score >0.30 and the T-cell mediated rejection score <0.20 was used for selection of transcripts for the extended list, for further confirmation.

### Derivation Phase

In the derivation phase, the multivariate combination of transcripts that lead to the best model accuracy was identified, based on the extended list of transcripts obtained in the discovery phase. This identification of the multigene signature was done by ranking a combination of genes according to the C-statistic of logistic regression models trained on this combination and estimated under a 3-folds cross validation. The number of evaluations to test was equal to 2ⁿ where n was the number of transcripts available in the restricted list and corresponds to all the combinations of groups of all sizes from 1 to n. During this process, and in order to rank the signatures, it was assumed that a relevant group of transcripts leads to a good accuracy. The measure used to rank a given combination of variables was the AUC value reached by a logistic regression model trained on this combination and estimated under a 3-folds cross validation. Instead of identifying the best combination as the final signature, the combinations obtained by the top K models that were integrated were identified. Let assume that the combinations are ranked according to the model accuracy (AUC) and let be bₖᵢ, a Boolean value that indicates if the biomarkers indexed by i is selected in the combination k bₖᵢ∈ [symbol] 0; 1 [symbol] , k∈ [symbol] 1 . . . K [symbol] , i∈ [symbol] 1 . . . n [symbol] . Let f_{Ki} be the frequency of selection of variable i among the top K combinations:

$${f}_{Ki} = \frac{1}{K} {\sum }_{k = 1}^{K} {b}_{ki} .$$

Then the frequency profile fₖᵢ was followed for the variable i and seen whether it is involved in the best models and combinations. Let

$${K}_{c} = {10}^{\frac{n}{10}}$$

be the cut-off value corresponding to the number of top combinations considered to identify the signatures (where n is the number of variables). The subset of variables to consider for the signature was then composed by the indexes i of the combination for which

$$\frac{1}{{K}_{c}} {\sum }_{k = 1}^{{K}_{c}} {b}_{ki} \geq α$$

where α was set to 0.6 for the study. The best multigene signature was then used to build a multivariable logistic regression model in a nested-ross validation approach on the derivation cohort. The ensuing logistic regression model was then locked and represented the final multigene assay.

### Analysis

In the discovery phase, RMA-normalized mRNA expression data of the 117 peripheral blood samples and 95 biopsies were analyzed in a statistical pipeline developed under the R framework in an extension of the biosigner R package as developed for this study [Rinaudo et al. (2016) Front Mol Biosci 3, 26], with addition of Elastic-Net and Shrunken Centroids multivariate methods to the SPLS, Random Forrest and SVM-RFE multivariate methods already available in the biosigner package. The constructed statistical pipeline and determination of a multivariate score for antibody-mediated rejection (ABMR score) and T-cell mediated rejection (TCMR score) is given in detail below. A multivariate score >0.25 was considered as specific for antibody-mediated and/or T-cell mediated rejection. Ingenuity Pathway Analysis (IPA, Build: 478438M Content version: 44691306) was used for canonical pathway enrichment analysis.

In the derivation phase, the multivariate combination of transcripts that lead to the best model accuracy was identified, based on the extended list of transcripts obtained in the discovery phase. This identification of the multigene signature was done by ranking a combination of genes according to the C-statistic of logistic regression models trained on this combination and estimated under a 3-folds cross validation.

Instead of identifying the best combination as the final multigene signature, the combinations obtained by the top K models were integrated. The best multigene signature was then used to build a multivariable logistic regression model in a nested-cross validation approach on the derivation cohort. The ensuing logistic regression model was then locked and represented the final multigene assay.

The diagnostic performance of the locked multigene signature and logistic regression model calculated in the derivation phase was then evaluated on the validation cohort. Receiver Operating Characteristic (ROC) curves were used to evaluate the C-statistic (area under the curve, AUC) of the multigene assay. The optimal marker threshold was calculated by the Youden index. Arbitrarily low and high thresholds with respectively high negative predictive value and high positive predictive value were also defined. Finally, sensitivity analyses were performed to evaluate the performance of the marker in specific clinical situations. The diagnostic value that the marker added to a reference clinical risk model (multivariable model of diagnosis of antibody-mediated rejection consisting of graft functional data and circulating donor-specific anti-HLA antibodies) was evaluated using decision curve analysis [Vickers et al. (2006) Elkin EB. 26, 565-574; Vickers et al. (2016) BMJ 352, i6]. For variance analysis of continuous clinical variables in different groups, non-parametric Wilcoxon-Mann-Whitney U, non-parametric ANOVA and parametric one-way ANOVA were used. Dichotomous variables were compared using the chi-square test. R [The R Project for Statistical Computing [Internet]. [cited 2018 Oct. 22]. Available from: https://www.r-project.org/], SAS (version 9.4; SAS institute, Cary, N.C.) and GraphPad Prism (version 7; GraphPad Software, San Diego, Calif.) were used for data presentation. Normalized signal intensities and .CEL files of the transcriptomic data will be made available in the NIH Gene Expression Omnibus http://www.ncbi.nlm.nih.gov/geo upon publication.

### Diagnostic Accuracy of the 8-Gene Assay in the Validation Cohort

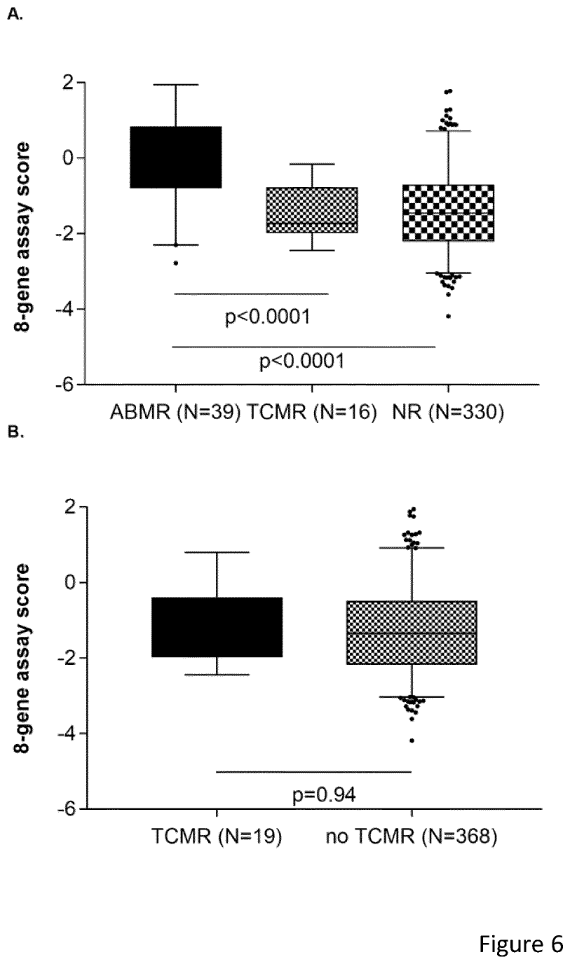

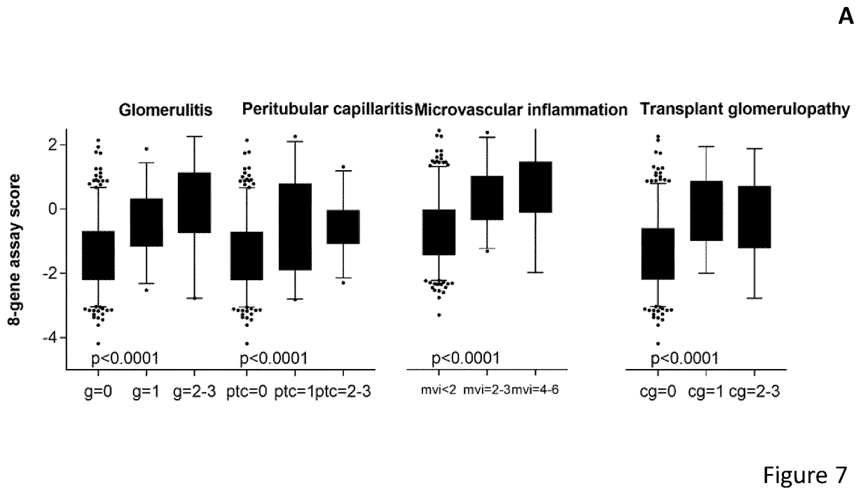

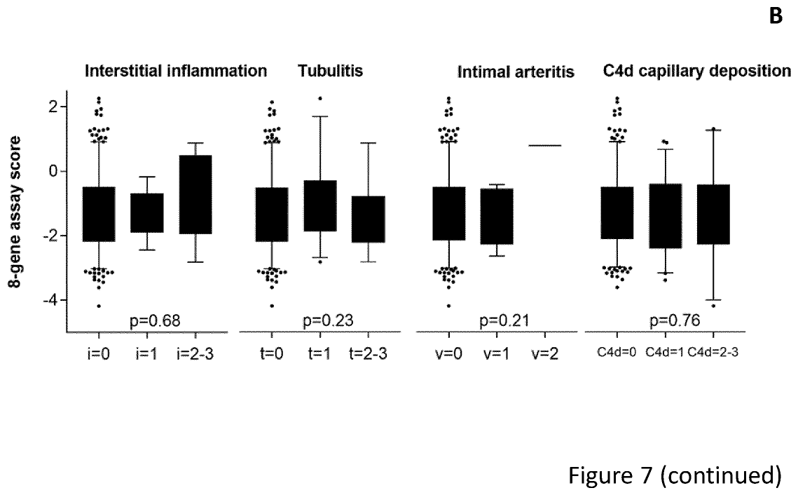

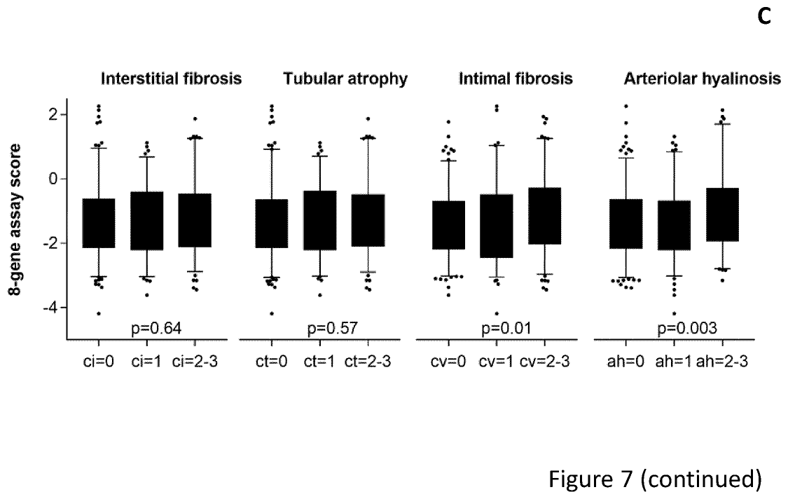

The 8-gene signature and logistic regression model built on the derivation cohort were then evaluated on the 387 samples collected in the cross-sectional study, which contained 41 cases with antibody-mediated rejection (10.6%), which represents the natural prevalence of this phenotype in the cohort of biopsies performed at the participating centres. Diagnostic accuracy of the 8-gene assay was 79.9% (95% CI, 72.6 to 87.2; p<0.001)(FIG. 2). When the diagnostic performance for discrimination was evaluated between pure antibody-mediated rejection (N=38) and pure T-cell mediated rejection (N=16), the 8-gene assay reached an accuracy of 82.2% (95% CI, 70.7 to 93.8; p=0.001), and 79.3% (95% CI, 71.6 to 86.9; p<0.001) for discrimination between pure antibody-mediated rejection (N=38) and absence of rejection (N=330). This 8-gene assay was not diagnostic for T-cell mediated rejection (FIG. 6).

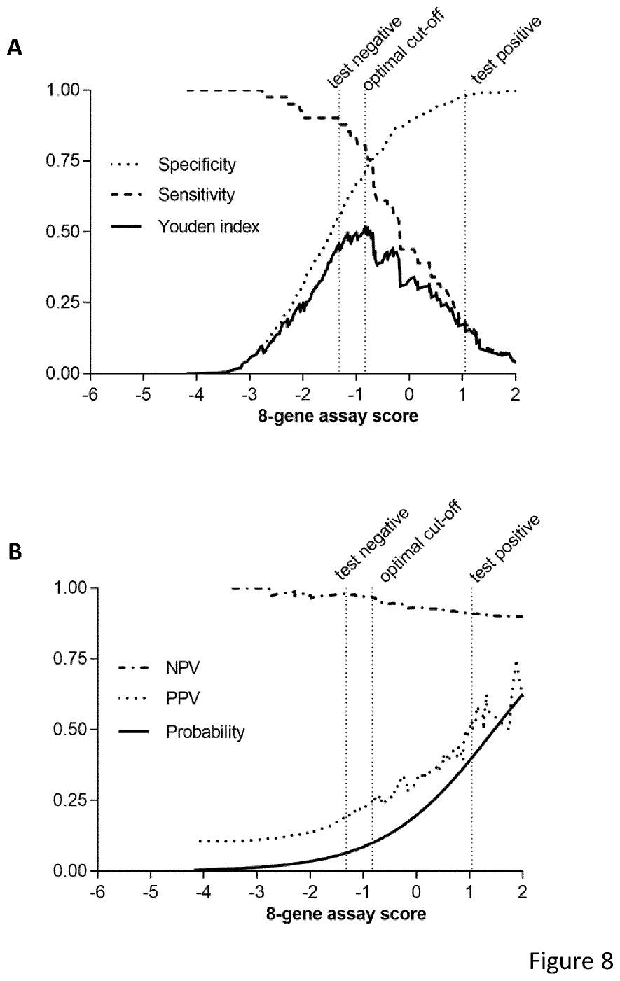

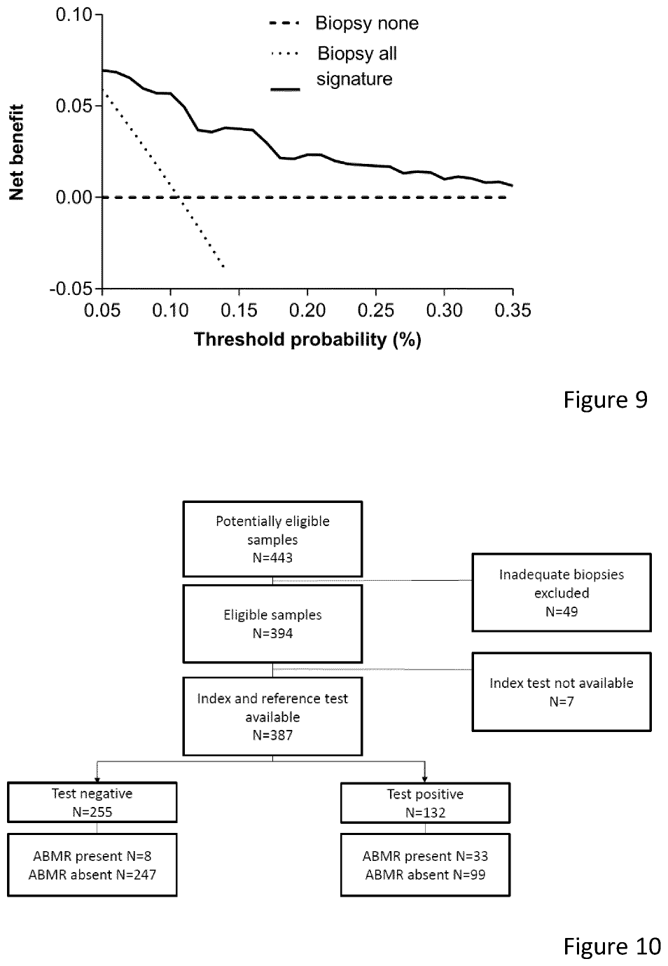

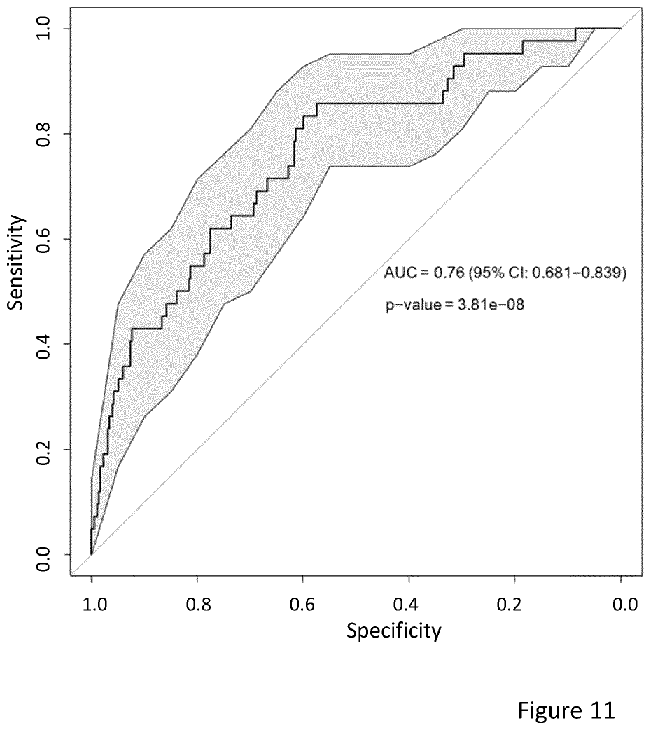

Next, the best cut-off values of the 8-gene assay in the validation cohort was determined since this cohort contained realistic disease prevalence (FIG. 8). The optimal cut-off (at the highest Youden index) for the 8-gene biomarker was −1.215, with an associated sensitivity of 80.5%, specificity of 71.4%, positive predictive value of 25.0% and negative predictive value of 96.9% (Table 5; FIG. 8).

TABLE 5
Diagnostic performance of the 8-gene marker for non-invasive diagnosis of
antibody-mediated rejection in the independent validation cohort (N = 387).

| | | | | Positive | Negative |
|---|---|---|---|---|---|
| | Diagnostic | | | Predictive | Predictive |
| | Accuracy | Sensitivity | Specificity | Value | Value |
| Test, Threshold and Cohort | % (95% Cl) | % | % | % | % |
| 8-gene marker | | | | | |
| All biopsies (N = 387) | 79.9% (72.7- | | | | |
| | 87.2) | | | | |
| Low threshold^{§} (−1.32) | | 90.2% | 55.8% | 19.5% | 98.0% |
| (Test negative) | | | | | |
| Optimal threshold* (−0.83) | | 80.5% | 71.4% | 25.0% | 96.9% |
| High threshold^{§} (1.04) | | 19.5% | 98.0% | 53.3% | 91.1% |
| Early biopsies <1 year (N = 207) | 90.9% (85.3- | | | | |
| | 96.4) | | | | |
| Low threshold (−1.32) | | 100% | 62.4% | 11.9% | 100% |
| (Test negative) | | | | | |
| Optimal threshold (−0.83) | | 100% | 75.6% | 17.2% | 100% |
| High threshold (1.04) | | 20.0% | 97.5% | 28.6% | 96.0% |
| Late biopsies >1 year (N = 180) | 73.5% (63.6- | | | | |
| | 83.4) | | | | |
| Low threshold (−1.32) | | 87.1% | 46.3% | 25.2% | 94.5% |
| (Test negative) | | | | | |
| Optimal threshold (−0.83) | | 74.2% | 65.1% | 30.7% | 92.4% |
| High threshold (1.04) | | 19.4% | 98.0% | 66.7% | 85.4% |
| Biopsies at time of graft dysfunction | 75.3% (64.9- | | | | |
| (N = 134) | 85.8) | | | | |
| Low threshold (−1.32) | | 86.7% | 49.0% | 32.9% | 92.7% |
| (Test negative) | | | | | |
| Optimal threshold (−0.83) | | 83.3% | 63.5% | 39.7% | 93.0% |
| High threshold (1.04) | | 23.3% | 96.2% | 63.7% | 81.3% |
| Biopsies at time of stable graft | 83.4% (75.4- | | | | |
| function (N = 253) | 91.3) | | | | |
| Low threshold (−1.32) | | 100% | 58.3% | 9.8% | 100% |
| (Test negative) | | | | | |
| Optimal threshold (−0.83) | | 72.7% | 74.4% | 11.4% | 98.4% |
| High threshold (1.04) | | 9.1% | 98.3% | 20.0% | 96.0% |
| Early (<1 year) biopsies at time of | 88.4% (77.7- | | | | |
| graft dysfunction (N = 55) | 99.2) | | | | |
| Low threshold (−1.32) | | 100% | 62% | 20.8% | 100% |
| (Test negative) | | | | | |
| Optimal threshold (−0.83) | | 100% | 74% | 27.8% | 100% |
| High threshold (1.04) | | 20% | 98% | 50.0% | 92.5% |
| (Test positive) | | | | | |

Routine post-transplant biomarkers (all biopsies N = 387)

| | | | | |
|---|---|---|---|---|
| eGFR | 67.8% (57.4- | — | — | — |
| (mL/min/1.73 m²) | 78.2) | | | |
| Proteinuria | 75.9% (66.6- | — | — | — |
| (g/g creatinine) | 85.1) | | | |
| Donor-specific antibodies | 65.3%(57.4- | — | — | — |
| (presence/absence) | 73.2) | | | |

*The optimal threshold for the ROC curve was calculated by Youden's index (using the maximum summation of the sensitivity plus specificity minus 1) in the full cohort.
^{§}Low and high threshold were arbitrarily selected in the full cohort.

### Sensitivity Analysis

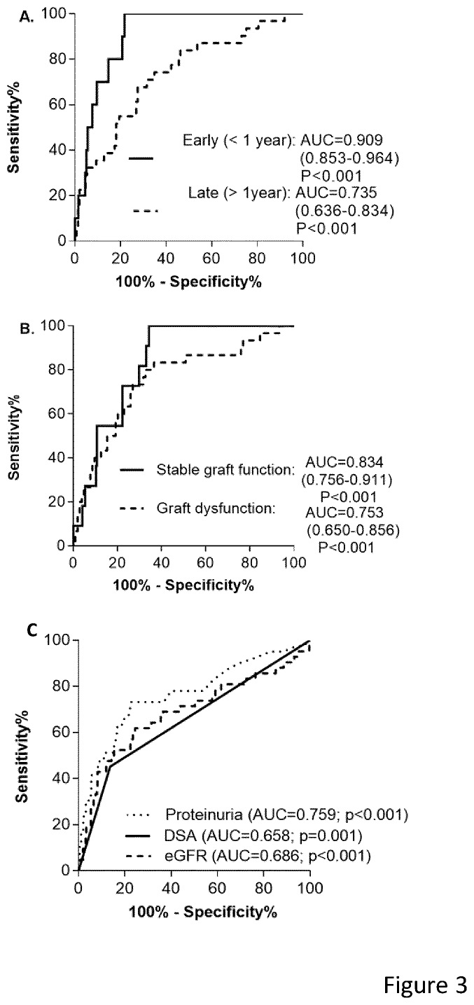

The 8-gene assay retained accuracy for antibody-mediated rejection in patients with stable graft function and at time of graft dysfunction, within the first year and also later after transplantation (Table 5, FIG. 3). In early biopsies and biopsies at time of stable graft function, the signature allowed to rule out ongoing antibody-mediated rejection with high negative predictive value.

Correlation of the 8-Gene Assay with Histological and Clinical Variables

The 8-gene assay correlated with graft functional parameters like eGFR and proteinuria, and with histological lesions diagnostic for antibody-mediated rejection like glomerulitis, peritubular capillaritis, microvascular inflammation and transplant glomerulopathy in the validation cohort (Table 6).

TABLE 6
Correlation of signature with clinical and histological variables
using Spearman correlation in the validation cohort (N = 387).

| | Correlation | 95% confidence | |
|---|---|---|---|
| Variable | coefficient | interval | P value |
| Microvascular inflammation | 0.31 | 0.21-0.39 | <0.001 |
| Peritubular capillaritis | 0.28 | 0.18-0.37 | <0.001 |
| Glomerulitis | 0.29 | 0.20-0.38 | <0.001 |
| Intimal arteritis | 0.03 | −0.07-0.13 | 0.58 |
| Peritubular C4d deposition | −0.02 | −0.12-0.08 | 0.74 |
| Tubulitis | 0.04 | −0.06-0.14 | 0.39 |
| Interstitial inflammation | 0.02 | −0.08-0.12 | 0.64 |
| Arteriolar hyalinosis | 0.14 | 0.04-0.23 | 0.007 |
| Transplant glomerulopathy | 0.21 | 0.11-0.30 | <0.001 |
| Tubular atrophy | 0.06 | −0.04-0.16 | 0.22 |
| Vascular intimal thickening | 0.13 | 0.03-0.23 | 0.009 |
| Interstitial fibrosis | 0.06 | −0.04-0.16 | 0.26 |
| Interstitial fibrosis/ | 0.06 | −0.04-0.16 | 0.24 |
| tubular atrophy | | | |
| Polyomavirus-associated | 0.03 | −0.07-0.13 | 0.58 |
| nephropathy | | | |
| Glomerulonephritis | −0.04 | −0.14-0.06 | 0.41 |
| Donor-specific HLA | 0.03 | −0.07-0.13 | 0.55 |
| antibodies | | | |
| Proteinuria | 0.15 | 0.05-0.25 | 0.003 |
| eGFR (MDRD) | −0.13 | −0.23-−0.03 | 0.01 |
| Recipient age at time of | 0.02 | −0.08-0.12 | 0.68 |
| biopsy | | | |
| Donor age | 0.02 | −0.08-0.12 | 0.72 |
| Gender recipient | 0.10 | 0.00-0.20 | 0.04 |
| (reference = male) | | | |
| Gender donor | 0.04 | −0.06-0.14 | 0.47 |
| (reference = male) | | | |

There was no correlation with histological lesions of T-cell mediated rejection. The 8-gene biomarker did not associate with diagnosis of glomerulonephritis, polyomavirus associated nephropathy or interstitial fibrosis with tubular atrophy.

Comparison with Traditional Biomarkers and Added Clinical Value of the 8-Gene Assay

The 8-gene assay associated with diagnosis of antibody-mediated rejection, independent of traditional factors associating with antibody-mediated rejection (female gender, presence of donor-specific antibodies and proteinuria) (Table 7).

TABLE 7
Univariate and multivariate logistic regression analysis of clinical variables
for diagnosis of antibody-mediated rejection in the validation cohort (N = 387).

| | | | Multivariate | | Multivariate | |
|---|---|---|---|---|---|---|
| | | | excluding 8-gene | | including 8-gene | |
| | Univariate | | marker | | marker | |
| | Odds ratio | | Odds ratio | | Odds ratio | |
| Variable | (95% CI) | P value | (95% CI) | P value | (95% CI) | P value |
| eGFR (MDRD) | 0.97 (0.95- | 0.002 | 0.98 (0.95- | 0.16 | 0.99 (0.96- | 0.39 |
| | 0.99) | | 1.00) | | 1.02) | |
| Proteinuria (g/g | 1.99 (1.50- | <0.001 | 1.55 (1.15- | 0.005 | 1.72 (1.21- | 0.002 |
| creatinine)* | 2.65) | | 2.11) | | 2.43) | |
| Recipient age (at | 0.97 (0.95- | 0.002 | 1.01 (0.98- | 0.74 | 1.01 (0.98- | 0.48 |
| time of Tx) | 0.99) | | 1.04) | | 1.05) | |
| Donor age * | 0.96 (0.94- | <0.001 | 0.97 (0.94- | 0.06 | 0.96 (0.93- | 0.01 |
| | 0.98) | | 1.00) | | 0.99) | |
| Recipient gender | 2.57 (1.30- | 0.007 | 2.52 (1.11- | 0.03 | 2.32 (0.95- | 0.07 |
| (reference = M)* | 5.07) | | 5.73) | | 5.68) | |
| Donor gender | 0.57 (0.28- | 0.11 | | | | |
| (reference = M)* | 1.13) | | | | | |
| Days after | 1.00 (1.00- | <0.001 | 1.00 (1.00- | 0.02 | 1.00 (1.00- | 0.13 |
| transplantation | 1.00) | | 1.00) | | 1.00) | |
| Cold ischemia | 1.02 (0.98- | 0.37 | | | | |
| time (hours)* | 1.07) | | | | | |
| Donor-specific | 5.09 (2.55- | <0.001 | 5.43 (2.37- | <0.001 | 7.50 (2.82- | <0.001 |
| antibodies | 10. 18) | | 12.47) | | 20.0) | |
| 8-gene marker | 2.61 (1.91- | <0.001 | | | 2.84 (1.88- | <0.001 |
| | 3.55) | | | | 4.27) | |

In multivariate analysis, a clinical reference model with routine clinical parameters (eGFR, proteinuria and DSA) reached accuracy for diagnosis of antibody-mediated rejection of 78.5% (95% CI, 69.4 to 87.6). When the 8-gene marker was added to this reference model, the accuracy for diagnosis of antibody-mediated rejection significantly increased to 87.8% (95% CI, 82.6 to 93.0; p=0.003) (Table 5). Decision curve analysis confirmed the net benefit of using the 8-gene assay for diagnosis of antibody-mediated rejection, and also of adding the 8-gene assay to routine markers (donor-specific antibodies, estimated glomerular filtration rate and proteinuria), across the range of probability thresholds between 0% and 30% (FIG. 10).

The samples used for the final calculation of the 8-gene marker (N=183) were unique samples per patient, taken from a larger group of samples collected in this same patient population (N=183 patients with 259 biopsies). For the final marker calculation, only the first biopsy for each patient was included.

When the signature selection pipeline, and logistic regression analysis was performed on these 265 biopsies, slightly different signatures and results were obtained.

From table 8, it can be concluded that at least the following genes should be included in the mRNA gene signature for ABMR: FCGR1A, FCGR1B, CXCL10 and TIMP1.

The diagnostic accuracy of the minimal 4-gene panel is slightly less than of the 8-gene panel, but still highly significant and clinically relevant, with a ROCAUC of 0.76 (95% CI: 0.681-0.839; p-value=3.81e-08) in the independent validation cohort.

TABLE 8
Gene signatures for differentiation between samples with vs. without ABMR.

| 8-gene signature | 7-gene signature | Minimal signature | Full Signature |
|---|---|---|---|
| 1st biopsies | All biopsies (N = 259) | Overlap 8-gene and | All genes significant |
| (N = 183) | | 7-gene signature | in multivariate or |
| | | | univariate analysis |
| CXCL10 | FCGR1A | FCGR1A | FCGR1A |
| GBP1 | FCGR1B | FCGR1B | FCGR1B |
| IL15 | CXCL10 | CXCL10 | CXCL10 |
| FCGR1A | TIMP1 | TIMP1 | TIMP1 |
| FCGR1B | FAS | | FAS, MAP2K5, GBP1, |
| GBP4 | MAP2K5 | | IL15, GBP4, KLRC1, |
| KLRC1 | KLRD1 | | CD274, CD38, |
| TIMP1 | | | CD46, CRTAM, |
| | | | CXCL9, CXCL11, |
| | | | ETV7, IFIH1, GBP2, |
| | | | GBP5, KCNJ2, |
| | | | MAP3K8, SLAMF7, |
| | | | STX11, TAP2 |

1. A method for diagnosing graft rejection other than T cell mediated graft rejection or determining the risk of developing graft rejection other than T cell mediated graft rejection in a subject who has received a solid organ transplant, the method comprising the steps of:
measuring in a peripheral blood sample from said subject the RNA expression levels of a set of genes comprising at least CXCL10 (C-X-C Motif Chemokine Ligand 10), FCGR1A (Fragment Of IgG Receptor Ia), FCGR1B Fragment Of IgG Receptor Ib) and TIMP1 (TIMP Metallopeptidase Inhibitor 1),
determining, from the measured expression levels of said set of genes, an expression profile,
comparing the determined expression profile of said subject with a reference expression profile, and
determining, based on the comparing, whether said subject is experiencing said graft rejection or has an increased risk of developing said graft rejection.

2. The method according to claim 1, wherein the set of genes further comprises one or more of GBP4 (Guanylate Binding Protein 4), KLRC1 (Killer Cell Lectin Like Receptor C1), GBP1 (Guanylate Binding Protein 1) and IL 15 (Interleukin 15).

3. The method according to claim 1, wherein the measured expression levels are normalized against housekeeping genes.

4. The method according to claim 1, wherein the graft rejection other than T cell mediated graft rejection is selected from the group consisting of antibody mediated rejection, changes suspicious of antibody-mediated rejection, and mixed T cell and antibody mediated rejection.

5. The method according to claim 1, wherein the graft rejection other than T cell mediated graft rejection is antibody mediated rejection.

6. The method according to claim 1, wherein the solid organ transplant is a kidney transplant.

7. The method according to claim 1, wherein, for determining of the expression profile, a weight factor is attributed to the quantitative expression level of each gene, wherein the weight factor of CXCL10<GBP4<KRLC1<FCGR1A and wherein the weight factor of GBP1<FCGR1B<IL15<TIMP1.

8. The method according to claim 7, wherein the weight factor of GBP1<CXCL10<GBP4<KRLC1<FCGR1B<IL15<TIMP1<FCGR1A.

9. The method according to claim 1, wherein a linear regression algorithm is used to compare the expression profile of said subject with a reference expression profile.

10. The method according to claim 9, wherein the linear regression algorithm is represented by the formula
log(odds ABMR)=“constant value”+a*ΔCq_CXCL10+b*ΔCq_GBP1+c*ΔCq_IL15+d*ΔCq_FCGR1A+e*ΔCq_FCGR1B+f*ΔCq_GBP4+g*ΔCq_KLRC1+h*ΔCq_TIMP1,
wherein a value of log (odds ABMR) above −0.83 is indicative of rejection other than T cell mediated rejection,
wherein “constant value” is −2.52±0.25, a is −0.25±0.025, b is 0.086±0.0086, c is 0.76±0.076, d is −1.21±0.12, e is +0.45±0.045, f is −0.31±0.031, g−0.40±0.04 and is h is 1.03±0.1,
and wherein ΔCq of a gene corresponds to the mean delta-Cq of each gene, which is the difference between the measured Cq value of each gene and the mean Cq value of three housekeeping genes.

11. The method according to claim 1, wherein the RNA expression levels are determined by quantitative PCR amplification.

12. A method for distinguishing, in a subject who has received a kidney transplant, between antibody mediated rejection and T cell mediated rejection, comprising the steps of:
measuring in a peripheral blood sample from said subject the RNA expression level of a set of genes comprising at least CXCL10, FCGR1A, FCGR1B and TIMP1,
comparing the measured RNA expression levels of each genes in the set with the RNA expression level of at least one reference housekeeping gene,
wherein a high expression level of CXCL10 and FCGR1A compared to the expression level of the least one reference gene, and a low expression level of FCGR1B and TIMP1 compared to the expression level of the least one reference gene is indicative of antibody mediated rejection.

13. (canceled)

14. A kit for in vitro diagnosis of kidney transplant rejection other than T cell mediated rejection comprising:
(i) a nucleic acid probe or set of nucleic acid probes for determining the expression level of at least CXCL10, FCGR1A, FCGR1B and TIMP1, and optionally a nucleic acid probe or set of nucleic acid probes for determining the expression level of GBP4, KLRC1, GBP1 and IL 15, and
(ii) optionally a nucleic acid probe or set of nucleic acid probes for determining the expression level of housekeeping genes,
wherein the kit comprises no more than 50 nucleic acid probes for determining the expression level of a gene that does not belong to the genes of (i) and that is not a housekeeping gene,
or
wherein the kit comprises no more than 50 sets of nucleic acid probes for determining the expression of gene that does not belong to the genes of (i) and that is not a housekeeping gene.

15. The kit according to claim 14, further comprising instructions for use in the form of a formula for calculating an expression profile and for calculating the odds of rejection other than T cell mediated rejection.
